(19) United States
(12) Patent Application Publication
(10) Pub. No.: US 2024/0299386 A1
(43) Pub. Date: Sep. 12, 2024

(54) Novel imidazole derivatives having protein kinase inhibitory activity and uses thereof

(71) Applicant: INDUSTRY-UNIVERSITY COOPERATION FOUNDATION HANYANG UNIVERSITY ERICA CAMPUS, Ansan-si Gyeonggi-do (KR)

(72) Inventors: Jung-Mi Hah, Seoul (KR); Joon Hong Jun, Ansan-si Gyeonggi-do (KR); Ji Hyun Baek, Ansan-si Gyeonggi-do (KR)

(21) Appl. No.: 18/571,331

(22) PCT Filed: Aug. 16, 2022
(86) PCT No.: PCT/KR2022/012173

§ 371 Date: Dec. 18, 2023

(30) Foreign Application Priority Data
Aug. 18, 2021 (KR) 10-2021-0108424

(51) Int. Cl. A61K 31/506; C07D 401/14; C07D 403/14; C07D 405/14

(52) U.S. Cl. CPC A61K 31/506; C07D 401/14; C07D 403/14; C07D 405/14

(57) ABSTRACT

The present invention relates to: a novel imidazole derivative compound; and isomer thereof or a pharmaceutically acceptable salt thereof; and a pharmaceutical composition comprising same. The imidazole derivative compound according to the present invention exhibits selective inhibitory activity against JNK, particularly JNK3, and thus can be used as a pharmaceutical composition for use in preventing and treating degenerative brain diseases (degenerative brain diseases including dementia of Alzheimer's type dementia and Parkinson's disease).

### TECHNICAL FIELD

The present invention relates to a novel imidazole derivative having inhibitory activity against the protein kinase, c-Jun N-terminal kinase 3 (JNK3), and uses thereof.

The present invention was made under Project no. NRF-2019M3A9A8066500 under the support of the Ministry of Science and ICT (MSIT) of the Republic of Korea (2017Y); the research management agency for this project is “National Research Foundation of Korea,” the research project name is “Original Technology Development Project/Bio-medical Technology Development Project/New Drug Target Discovery and Verification Project,” the research project title is “Validation of INK inhibitor with effects of inhibiting nerve cell death and improving cognitive function as a therapeutic agent for Alzheimer's dementia”, and the research period is “2020.03.01.-2020.12.31.”

In addition, the present invention was made under Project no. NRF-2020R1A6A1A03042854 under the support by the Ministry of Education (2020Y); the research management agency for this project is “National Research Foundation of Korea,” the research project name is “Basic Research Project in the Field of Science and Engineering/Basic Research Infrastructure Construction Project/University Key Research Institute Support Project—Key Research Institute Support (Science and Engineering Fields),” the research project title is “Pharmaceutical technology research institute,” and the research period is “2021.03.01.-2022.02.28.”

This application claims priority to and the benefit of Korean Patent Application No. 10-2021-0108424, filed on Aug. 18, 2021, the disclosure of which is incorporated herein by reference in its entirety.

### BACKGROUND ART

In the case of Alzheimer's dementia, which is manifested by fatal dysfunction of the nervous system and motor impairment, due to a lack of understanding of the pathological phenomenon, existing therapeutic agents are mainly acetylcholinesterase inhibitors that alleviate the worsening of symptoms. According to continuous research on pathological phenomena, recently, with the development of therapeutic agents for degenerative brain diseases, there has been a so-called paradigm shift that moves from symptom alleviation to the development of a novel target protein inhibitor, and at the center of this, there are protein kinases, which are intracellular signaling proteins that control the apoptosis and necrosis of nerve cells.

In a representative signaling system, the MAPK pathway, which controls cell apoptosis, unlike approximately 10 similar isozymes, it is known that the c-Jun N-terminal kinase 3 (JNK3) isoform is concentrated in brain tissue and thus is deeply related to the apoptosis of nerve cells, and is distributed in pyramidal neurons (yellow) located in the CA1 and CA2 parts of the cerebral cortex and hippocampus, causing apoptosis due to overexpression of c-Jun, which is one of the intraneuronal substances, the degree of phosphorylation by JNK3, and the hyperactivation of JNK3.

First, JNK3 phosphorylates and activates amyloid precursor protein (APP), which is a precursor of Aβ as the main cause of Alzheimer's dementia, to be located in the cell membrane and to promote conversion into Aβ42. Particularly, the endocytosis of phosphorylated APP in this process is known to be a critical step in Aβ42 production. Second, it is noteworthy that Tau, which is the main ingredient of NFT found in brain cells of Alzheimer patients, is also phosphorylated by JNK. In addition, it was reported that neurodegeneration induced by APP/Aβ is attenuated (neuroprotection) in JNK3 gene-deleted animal models (Tg2576/PSM146L transgenic mice), showing that JNK3 also mediates the production and toxicity of Aβ, and a JNK3 inhibitor can be effectively used as an AD therapeutic agent.

### DISCLOSURE

### Technical Problem

The present invention is provided to solve the above problems, in an effort to find novel materials having the potential to be developed as therapeutic agents for brain diseases such as Alzheimer's dementia, Parkinson's disease, and Huntington's disease, the present inventors tried to develop low-molecular inhibitors that target c-Jun N-terminal kinase (JNK3) to adjust its function, and thus identified a novel imidazole derivative exhibiting JNK3 inhibitory activity. Based on this, the present invention was completed.

The present invention is directed to providing a novel imidazole derivative exhibiting JNK3 inhibitory activity or a pharmaceutically acceptable salt thereof.

The present invention is also directed to providing a method of preparing a novel imidazole derivative exhibiting JNK3 inhibitory activity.

The present invention is also directed to providing a pharmaceutical composition for use in preventing or treating a degenerative brain disease, which includes the imidazole derivative or a pharmaceutically acceptable salt thereof as an active ingredient.

The present invention is also directed to providing a method of treating a degenerative brain disease, which includes administering the imidazole derivative or a pharmaceutically acceptable salt thereof to an individual or subject in need thereof.

The present invention is also directed to providing an imidazole derivative or a pharmaceutically acceptable salt thereof, which is used to treat a degenerative brain disease.

The present invention is also directed to providing uses of the imidazole derivative or a pharmaceutically acceptable salt thereof to be used in the preparation of a drug for treating a degenerative brain disease.

However, technical problems to be solved in the present invention are not limited to the above-described problems, and other problems which are not described herein will be fully understood by those of ordinary skill in the art from the following descriptions.

### Technical Solution

To achieve the purpose of the present invention, a compound of Formula 1 below or a pharmaceutically acceptable salt thereof is provided.

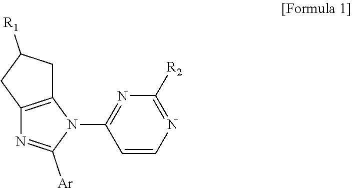

In Formula 1,
R₁ is selected from the group consisting of

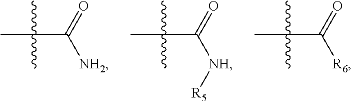

and C₁-C₆ alkyl,
R₂ is

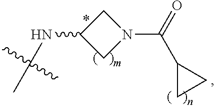

wherein
n is an integer of 1 to 4,
m is an integer of 2 and 3, and
the carbon indicated by * is a chiral carbon,
Ar is selected from the group consisting of

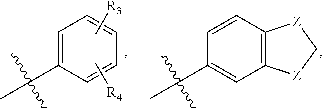

and naphthalenyl,
wherein R₃ and R₄ are each independently a halogen atom or halolalkyl,
R₅ is C₁-C₆ alkyl,
R₆ is alkoxy, and
Z is a carbon or oxygen atom.

In addition, the present invention provides a pharmaceutical composition for use in preventing or treating a degenerative brain disease, which includes the compound of Formula 1 above or a pharmaceutically acceptable salt thereof as an active ingredient.

In one embodiment of the present invention, the composition may inhibit the activity of JNK3.

### Advantageous Effects

A novel imidazole derivative or a pharmaceutically acceptable salt thereof according to the present invention exhibits excellent targeting and inhibitory activity against c-Jun N-terminal kinase 3 (JNK 3), and a pharmaceutical composition including the derivative can be effectively used for use in preventing and treating a degenerative brain disease, such as Alzheimer's dementia.

### DESCRIPTION OF DRAWINGS

FIG. 1 is a graph illustrating the inhibitory activity (%) when each of 50 types of protein kinases is treated with 22b (1 μM).

### MODES OF THE INVENTION

Hereinafter, the present invention will be described in detail.

The present invention provides a compound of Formula 1 or a pharmaceutically acceptable salt thereof.

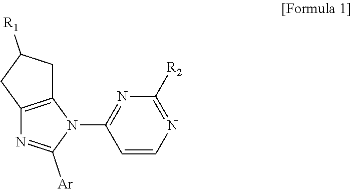

In Formula 1,
R₁ is selected from the group consisting of

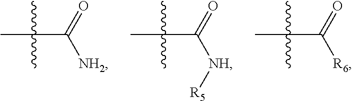

and C₁-C₆ alkyl,
R₂ is

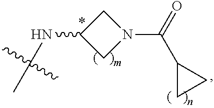

wherein
n is an integer of 1 to 4,
m is an integer of 2 and 3, and
the carbon indicated by * is a chiral carbon,
Ar is selected from the group consisting of

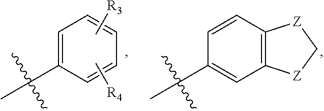

and naphthalenyl,
wherein R₃ and R₄ are each independently a halogen atom or halolalkyl,
R₅ is C₁-C₆ alkyl,
R₆ is alkoxy, and
Z is a carbon or oxygen atom.

Here, “chiral” is also called a chirality, a hand symmetry, an enantiomer or an optical isomer. Enantiomers are mirror images of each other.

Here, “alkyl” generally refers to a linear or branched saturated hydrocarbon group having a specified number of carbon atoms (e.g., 1 to 12 carbon atoms). Examples of alkyl groups include, but not limited to, methyl, ethyl, n-propyl, isopropyl, n-butyl, sec-butyl, isobutyl, tert-butyl, n-pentyl, n-hexyl, and n-heptyl. “Alkyl” may be attached to a parent group or a substrate at any ring atom provided that the attachment does not violate valence requirements. Likewise, an alkyl group or alkenyl group may include one or more non-hydrogen substituents provided that the attachment does not violate valence requirements.

Here, “alkoxy” refers to alkyl-O—, wherein the alkyl has been defined above. Examples of alkoxy groups include methoxy and ethoxy without limitation. “Alkoxy” may be attached to a parent group or a substrate at any ring atom provided that the attachment does not violate valence requirements. Likewise, the alkoxy group may include one or more non-hydrogen substituents provided that the attachment does not violate valence requirements.

Here, “halogen” encompasses elements belonging to Group 17 of the periodic table, including fluorine, chlorine, bromine, and iodine.

In one embodiment of the present invention, the compound of Formula 1 may be a compound with the structure of Formula 2 below.

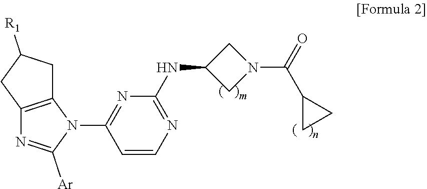

In Formula 2,
R₁ is selected from the group consisting of

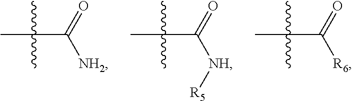

and C₁-C₆ alkyl,
n is an integer of 1 to 4,
m is an integer of 2 and 3,
Ar is selected from the group consisting of

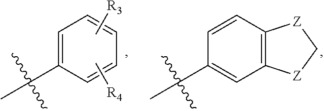

and naphthalenyl,
wherein R₃ and R₄ are each independently a halogen atom or haloalkyl,
R₅ is C₁-C₆ alkyl,
R₆ is alkoxy, and
Z is a carbon or oxygen atom.

In another embodiment of the present invention, the compound of Formula 1 may be a compound with the structure of Formula 3 below.

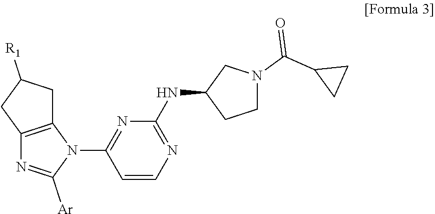

In Formula 3,
R₁ is selected from the group consisting of

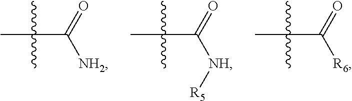

and C₁-C₆ alkyl,
Ar is selected from the group consisting of R₄,

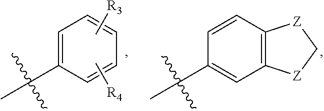

and naphthalenyl,
wherein R₃ and R₄ are each independently a halogen atom or haloalkyl,
R₅ is C₁-C₆ alkyl,
R₆ is alkoxy, and
Z is a carbon or oxygen atom.

According to still another embodiment of the present invention, the compound of Formula 1 may be a compound with the structure of Formula 4 below.

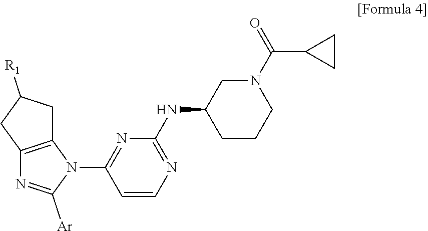

In Formula 4,
R₁ is selected from the group consisting of

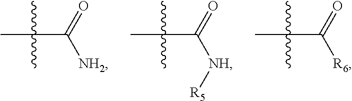

and C₁-C₆ alkyl,
Ar is selected from the group consisting of

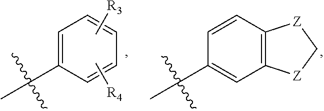

and naphthalenyl,
wherein R₃ and R₄ are each independently a halogen atom or haloalkyl,
R₅ is C₁-C₆ alkyl,
R₆ is alkoxy, and
Z is a carbon or oxygen atom.

According to yet another embodiment of the present invention, the compound of Formula 1 may be a compound with the structure of Formula 5 below.

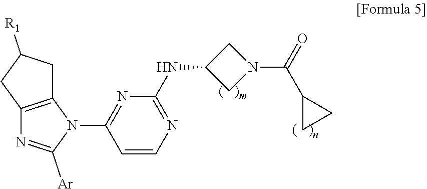

In Formula 5,
R₁ is selected from the group consisting of

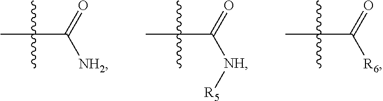

and C₁-C₆ alkyl,
n is an integer of 1 to 4,
m is an integer of 2 and 3,
Ar is selected from the group consisting of

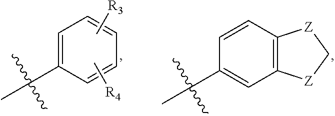

and naphthalenyl,
wherein R₃ and R₄ are each independently a halogen atom or haloalkyl,
R₅ is C₁-C₆ alkyl,
R₆ is alkoxy, and
Z is a carbon or oxygen atom.

According to yet another embodiment of the present invention, the compound of Formula 1 may be a compound with the structure of Formula 6 below.

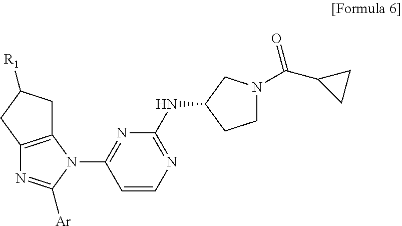

In Formula 6,
R₁ is selected from the group consisting of

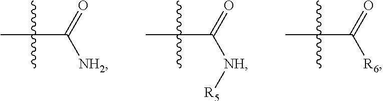

and C₁-C₆ alkyl,
Ar is selected from the group consisting of

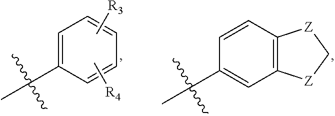

and naphthalenyl,
wherein R₃ and R₄ are each independently a halogen atom or haloalkyl,
R₅ is C₁-C₆ alkyl,
R₆ is alkoxy, and
Z is a carbon or oxygen atom.

According to yet another embodiment of the present invention, the compound of Formula 1 may be a compound with the structure of Formula 7 below.

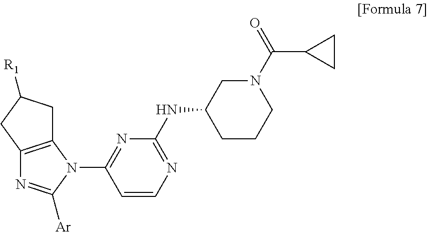

In Formula 7,
R₁ is selected from the group consisting of

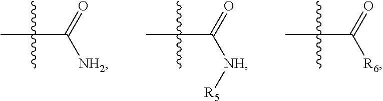

and C₁-C₆ alkyl,
Ar is selected from the group consisting of

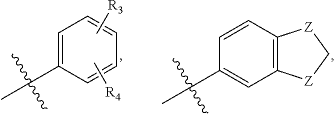

and naphthalenyl,
wherein R₃ and R₄ are each independently a halogen atom or haloalkyl,
R₅ is C₁-C₆ alkyl,
R₆ is alkoxy, and
Z is a carbon or oxygen atom.

According to yet another embodiment of the present invention, the imidazole compound of Formula 1 may be
ethyl 1-(2-(((S)-1-(cyclopropanecarbonyl)pyrrolidine-3-yl)amino)pyrimidin-4-yl)-2-(4-fluoro-3-(trifluoromethyl)phenyl)-1,4,5,6-tetracyclopenta[d]imidazol-5-carboxylate;
ethyl 1-(2-(((R)-1-(cyclopropanecarbonyl)pyrrolidine-3-yl)amino)pyrimidin-4-yl)-2-(4-fluoro-3-(trifluoromethyl)phenyl)-1,4,5,6-tetracyclopenta[d]imidazol-5-carboxylate;
ethyl 1-(2-(((S)-1-(cyclopropanecarbonyl)piperidin-3-yl)amino)pyrimidin-4-yl)-2-(4-fluoro-3-(trifluoromethyl)phenyl)-1,4,5,6-tetracyclopenta[d]imidazol-5-carboxylate;
ethyl 1-(2-(((R)-1-(cyclopropanecarbonyl)piperidin-3-yl)amino)pyrimidin-4-yl)-2-(4-fluoro-3-(trifluoromethyl)phenyl)-1,4,5,6-tetracyclopenta[d]imidazol-5-carboxylate;
1-(2-(((S)-1-(cyclopropanecarbonyl)pyrrolidine-3-yl)amino)pyrimidin-4-yl)-2-(4-fluoro-3-(trifluoromethyl)phenyl)-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(4-fluoro-3-(trifluoromethyl)phenyl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(4-fluoro-3-(trifluoromethyl)phenyl)-1,4,5,6-tetracyclopenta[d]imidazol-5-carboxamide;
1-(2-(((R)-1-(cyclopropanecarbonyl)piperidin-3-yl)amino)pyrimidin-4-yl)-2-(4-fluoro-3-(trifluoromethyl)phenyl)-1,4,5,6-tetracyclopenta[d]imidazol-5-carboxamide;
1-(2-(((S)-1-(cyclopropanecarbonyl)pyrrolidine-3-yl)amino)pyrimidin-4-yl)-2-(4-fluoro-3-(trifluoromethyl)phenyl)-N-methyl-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(4-fluoro-3-(trifluoromethyl)phenyl)-N-methyl-1,4,5,6-tetracyclopenta[d]imidazol-5-carboxamide;
1-(2-(((S)-1-(cyclopropanecarbonyl)piperidin-3-yl)amino)pyrimidin-4-yl)-2-(4-fluoro-3-(trifluoromethyl)phenyl)-N-methyl-1,4,5,6-tetracyclopenta[d]imidazol-5-carboxamide;
1-(2-(((R)-1-(cyclopropanecarbonyl)piperidin-3-yl)amino)pyrimidin-4-yl)-2-(4-fluoro-3-(trifluoromethyl)phenyl)-N-methyl-1,4,5,6-tetracyclopenta[d]imidazol-5-carboxamide;
ethyl 1-(2-(((S)-1-(cyclopropanecarbonyl)pyrrolidine-3-yl)amino)pyrimidin-4-yl)-2-(3,4-dichlorophenyl)-1,4,5,6-tetracyclopenta[d]imidazol-5-carboxylate;
ethyl 1-(2-(((R)-1-(cyclopropanecarbonyl)pyrrolidine-3-yl)amino)pyrimidin-4-yl)-2-(3,4-dichlorophenyl)-1,4,5,6-tetracyclopenta[d]imidazol-5-carboxylate;
ethyl 1-(2-(((S)-1-(cyclopropanecarbonyl)piperidin-3-yl)amino)pyrimidin-4-yl)-2-(3,4-dichlorophenyl)-1,4,5,6-tetracyclopenta[d]imidazol-5-carboxylate;
ethyl 1-(2-(((R)-1-(cyclopropanecarbonyl)piperidin-3-yl)amino)pyrimidin-4-yl)-2-(3,4-dichlorophenyl)-1,4,5,6-tetracyclopenta[d]imidazol-5-carboxylate;
1-(2-(((S)-1-(cyclopropanecarbonyl)pyrrolidine-3-yl)amino)pyrimidin-4-yl)-2-(3,4-dichlorophenyl)-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(3,4-dichlorophenyl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(3,4-dichlorophenyl)-1,4,5,6-tetracyclopenta[d]imidazol-5-carboxamide;
1-(2-(((R)-1-(cyclopropanecarbonyl)piperidin-3-yl)amino)pyrimidin-4-yl)-2-(3,4-dichlorophenyl)-1,4,5,6-tetracyclopenta[d]imidazol-5-carboxamide;
1-(2-(((S)-1-(cyclopropanecarbonyl)pyrrolidine-3-yl)amino)pyrimidin-4-yl)-2-(3,4-dichlorophenyl)-N-methyl-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(3,4-dichlorophenyl)-N-methyl-1,4,5,6-tetracyclopenta[d]imidazol-5-carboxamide;
1-(2-(((S)-1-(cyclopropanecarbonyl)piperidin-3-yl)amino)pyrimidin-4-yl)-2-(3,4-dichlorophenyl)-N-methyl-1,4,5,6-tetracyclopenta[d]imidazol-5-carboxamide;
1-(2-(((R)-1-(cyclopropanecarbonyl)piperidin-3-yl)amino)pyrimidin-4-yl)-2-(4-fluoro-3-(trifluoromethyl)phenyl)-N-methyl-1,4,5,6-tetracyclopenta[d]imidazol-5-carboxamide;
ethyl 1-(2-(((S)-1-(cyclopropanecarbonyl)pyrrolidine-3-yl)amino)pyrimidin-4-yl)-2-(naphthalen-2-yl)-1,4,5,6-tetracyclopenta[d]imidazol-5-carboxylate;
ethyl 1-(2-(((R)-1-(cyclopropanecarbonyl)pyrrolidine-3-yl)amino)pyrimidin-4-yl)-2-(naphthalen-2-yl)-1,4,5,6-tetracyclopenta[d]imidazol-5-carboxylate;
ethyl 1-(2-(((S)-1-(cyclopropanecarbonyl)piperidin-3-yl)amino)pyrimidin-4-yl)-2-(naphthalen-2-yl)-1,4,5,6-tetracyclopenta[d]imidazol-5-carboxylate;
ethyl 1-(2-(((R)-1-(cyclopropanecarbonyl)piperidin-3-yl)amino)pyrimidin-4-yl)-2-(naphthalen-2-yl)-1,4,5,6-tetracyclopenta[d]imidazol-5-carboxylate;
1-(2-(((S)-1-(cyclopropanecarbonyl)pyrrolidine-3-yl)amino)pyrimidin-4-yl)-2-(naphthalen-2-yl)-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(naphthalen-2-yl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(naphthalen-2-yl)-1,4,5,6-tetracyclopenta[d]imidazol-5-carboxamide;
1-(2-(((R)-1-(cyclopropanecarbonyl)piperidin-3-yl)amino)pyrimidin-4-yl)-2-(naphthalen-2-yl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(naphthalen-2-yl)-N-methyl-1,4,5,6-tetracyclopenta[d]imidazol-5-carboxamide;
1-(2-(((R)-1-(cyclopropanecarbonyl)piperidin-3-yl)amino)pyrimidin-4-yl)-2-(4-fluoro-3-(naphthalen-2-yl)-N-methyl-1,4,5,6-tetracyclopenta[d]imidazol-5-carboxamide;
ethyl 2-(benzo[d][1,3]dioxol-5-yl)-1-(2-(((S)-1-(cyclopropanecarbonyl)pyrrolidine-3-yl)amino)pyrimidin-4-yl)-1,4,5,6-tetracyclopenta[d]imidazol-5-carboxylate;
ethyl 2-(benzo[d][1,3]dioxol-5-yl)-1-(2-(((R)-1-(cyclopropanecarbonyl)pyrrolidine-3-yl)amino)pyrimidin-4-yl)-1,4,5,6-tetracyclopenta[d]imidazol-5-carboxylate;
ethyl 2-(benzo[d][1,3]dioxol-5-yl)-1-(2-(((S)-1-(cyclopropanecarbonyl)piperidin-3-yl)amino)pyrimidin-4-yl)-1,4,5,6-tetracyclopenta[d]imidazol-5-carboxylate;
ethyl 2-(benzo[d][1,3]dioxol-5-yl)-1-(2-(((R)-1-(cyclopropanecarbonyl)piperidin-3-yl)amino)pyrimidin-4-yl)-1,4,5,6-tetracyclopenta[d]imidazol-5-carboxylate;
2-(benzo[d][1,3]dioxol-5-yl)-1-(2-(((S)-1-(cyclopropanecarbonyl)pyrrolidine-3-yl)amino)pyrimidin-4-yl)-1,4,5,6-tetracyclopenta[d]imidazol-5-carboxamide;
2-(benzo[d][1,3]dioxol-5-yl)-1-(2-(((R)-1-(cyclopropanecarbonyl)pyrrolidine-3-yl)amino)pyrimidin-4-yl)-1,4,5,6-tetracyclopenta[d]imidazol-5-carboxamide;
2-(benzo[d][1,3]dioxol-5-yl)-1-(2-(((S)-1-(cyclopropanecarbonyl)piperidin-3-yl)amino)pyrimidin-4-yl)-1,4,5,6-tetracyclopenta[d]imidazol-5-carboxamide;
2-(benzo[d][1,3]dioxol-5-yl)-1-(2-(((S)-1-(cyclopropanecarbonyl)piperidin-3-yl)amino)pyrimidin-4-yl)-1,4,5,6-tetracyclopenta[d]imidazol-5-carboxamide;
2-(benzo[d][1,3]dioxol-5-yl)-1-(2-(((S)-1-(cyclopropanecarbonyl)pyrrolidine-3-yl)amino)pyrimidin-4-yl)-N-methyl-1,4,5,6-tetracyclopenta[d]imidazol-5-carboxamide;
2-(benzo[d][1,3]dioxol-5-yl)-1-(2-(((S)-1-(cyclopropanecarbonyl)pyrrolidine-3-yl)amino)pyrimidin-4-yl)-N-methyl-1,4,5,6-tetracyclopenta[d]imidazol-5-carboxamide;
ethyl 1-(2-(((S)-1-(cyclopropanecarbonyl)pyrrolidine-3-yl)amino)pyrimidin-4-yl)-2-(2,3-dihydrobenzofuran-5-yl)-1,4,5,6-tetracyclopenta[d]imidazol-5-carboxylate;
ethyl 1-(2-(((R)-1-(cyclopropanecarbonyl)pyrrolidine-3-yl)amino)pyrimidin-4-yl)-2-(2,3-dihydrobenzofuran-5-yl)-1,4,5,6-tetracyclopenta[d]imidazol-5-carboxylate;
ethyl 1-(2-(((S)-1-(cyclopropanecarbonyl)piperidin-3-yl)amino)pyrimidin-4-yl)-2-(2,3-dihydrobenzofuran-5-yl)-1,4,5,6-tetracyclopenta[d]imidazol-5-carboxylate;
ethyl 1-(2-(((R)-1-(cyclopropanecarbonyl)piperidin-3-yl)amino)pyrimidin-4-yl)-2-(2,3-dihydrobenzofuran-5-yl)-1,4,5,6-tetracyclopenta[d]imidazol-5-carboxylate;
1-(2-(((S)-1-(cyclopropanecarbonyl)pyrrolidine-3-yl)amino)pyrimidin-4-yl)-2-(2,3-dihydrobenzofuran-5-yl)-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(2,3-dihydrobenzofuran-5-yl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(2,3-dihydrobenzofuran-5-yl)-1,4,5,6-tetracyclopenta[d]imidazol-5-carboxamide;
1-(2-(((R)-1-(cyclopropanecarbonyl)piperidin-3-yl)amino)pyrimidin-4-yl)-2-(2,3-dihydrobenzofuran-5-yl)-1,4,5,6-tetracyclopenta[d]imidazol-5-carboxamide; or
1-(2-(((S)-1-(cyclopropanecarbonyl)piperidin-3-yl)amino)pyrimidin-4-yl)-2-(2,3-dihydrobenzofuran-5-yl)-N-methyl-1,4,5,6-tetracyclopenta[d]imidazol-5-carboxamide.

The present invention provides a pharmaceutical composition for use in preventing or treating a degenerative brain disease, which includes the derivative of Formula 1 or a pharmaceutically acceptable salt thereof.

According to yet another embodiment of the present invention, the degenerative brain disease may be Alzheimer's dementia, or Parkinson's disease.

According to yet another embodiment of the present invention, the composition may inhibit JNK3 activity.

Meanwhile, the compound of the present invention may be used in the form of a pharmaceutically acceptable salt, and as a salt, an acid addition salt formed by an acceptable free acid is useful.

The term “salt” used herein is an acid addition salt formed by a pharmaceutically acceptable free acid. The acid addition salt is obtained from inorganic acids such as hydrochloric acid, nitric acid, phosphoric acid, sulfuric acid, hydrobromic acid, hydroiodic acid, nitrous acid or phosphorous acid, aliphatic mono- and di-carboxylates, phenyl-substituted alkanoates, hydroxy alkanoates and alkane dioates, and non-toxic organic acids such as aromatic acids, aliphatic and aromatic sulfonic acids. Such pharmaceutically non-toxic salts include sulfates, pyrosulfates, bisulfates, sulfites, bisulfites, nitrates, phosphates, monohydrogen phosphates, dihydrogen phosphates, metaphosphates, pyrophosphate chlorides, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, butyne-1,4-dioates, hexane-1,6-dioates, benzoates, chlorobenzoates, methylbenzoates, dinitrobenzoates, hydroxylbenzoates, methoxybenzoates, phthalates, terephthalates, benzenesulfonates, toluenesulfonates, chlorobenzenesulfonates, xylenesulfonates, phenylacetates, phenylpropionates, phenylbutyrates, citrates, lactates, β-hydroxylbutyrates, glycolates, malates, tartrates, methanesulfonates, propanesulfonates, naphthalene-1-sulfonates, naphthalene-2-sulfonates, and mandelates.

The acid addition salt according to the present invention may be prepared by a conventional method, for example, dissolving a compound in an excess amount of acid aqueous solution, and precipitating the salt using a water-miscible organic solvent, such as methanol, ethanol, acetone, or acetonitrile. In addition, the acid addition salt according to the present invention may be prepared by evaporating a solvent or excess acid from the above mixture and then drying it, or by suction-filtering the precipitated salt.

In addition, a pharmaceutically acceptable metal salt may be prepared using a base. An alkali metal or alkaline earth metal salt is obtained by, for example, dissolving a compound in an excess of alkali metal hydroxide or alkaline earth metal hydroxide solution, filtering an insoluble compound salt, and evaporating and drying the filtrate. Here, it is pharmaceutically suitable to prepare a sodium, potassium, or calcium salt as a metal salt. The corresponding silver salt is obtained by reacting an alkali metal or alkaline earth metal salt with a suitable silver salt (e.g., silver nitrate).

In addition, the compound of the present invention includes not only a pharmaceutically acceptable salt but any salt, isomer, hydrate, and solvate that can be prepared by a conventional method.

As can be confirmed in the following examples, the compound of Formula 1 may be used as a JNK3 inhibitor, and as described in the background of the invention, it is widely known to those of ordinary skill in the art that the JNK3 inhibitor can be used to treat a degenerative brain disease.

The present invention provides a pharmaceutical composition for use in preventing or treating a degenerative brain disease, including the imidazole derivative of Formula 1 or a pharmaceutically acceptable salt thereof as an active ingredient, and more particularly, a pharmaceutical composition for use in preventing or treating a degenerative brain disease, uses of the imidazole derivative of Formula 1 or a pharmaceutically acceptable salt thereof for treating the disease, and a method of treating the disease, which includes administering a therapeutically effective amount of the compound of Formula 1 or a pharmaceutically acceptable salt thereof to a subject.

The term “prevention” used herein refers to all actions of inhibiting a degenerative brain disease or delaying the onset thereof by the administration of the pharmaceutical composition according to the present invention.

The term “treatment” used herein refers to all actions involved in alleviating or beneficially changing symptoms of a degenerative brain disease by the administration of the pharmaceutical composition according to the present invention.

The pharmaceutical composition according to the present invention may include a pharmaceutically acceptable carrier as well as an active ingredient. Here, the pharmaceutically acceptable carrier is conventionally used in drug formulation, and includes, but is not limited to, lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, and mineral oil. As well as the above components, the pharmaceutical composition according to the present invention may further include a lubricant, a wetting agent, a sweetening agent, a flavoring agent, an emulsifier, a suspending agent, or a preservative.

The pharmaceutical composition of the present invention may be administered orally or parenterally (e.g., intravenously, subcutaneously, intraperitoneally or topically), and preferably, intracerebrally, according to a desired method, and a dose of the pharmaceutical composition of the present invention may be suitably selected according to a patient's condition and body weight, severity of a disease, a dosage form, an administration route and duration by those of ordinary skill in the art.

The composition according to the present invention is administered at a pharmaceutically effective amount. The “pharmaceutically effective amount” used herein refers to an amount sufficient for treating a disease at a reasonable benefit/risk ratio applicable for medical treatment, and an effective dosage may be determined by parameters including the type and severity of a patient's disease, drug activity, sensitivity to a drug, administration time, an administration route and an excretion rate, the duration of treatment and drugs simultaneously used, and other parameters well known in the medical field. The composition according to the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without side effects, and such a dose may be easily determined by one of ordinary skill in the art.

Specifically, the effective amount of the pharmaceutical composition of the present invention may vary depending on a patient's age, sex, condition and body weight, an absorption rate of the active ingredient in the body, an inactivation rate, an excretion rate, a type of disease, or a drug used in combination, and may be generally administered at 0.0001 to 1000 mg/kg, and preferably, 0.001 to 500 mg/kg of body weight daily or every other day, or divided into one or three daily administrations. However, the effective amount may vary depending on an administration route, the severity of obesity, sex, body weight or age, and therefore, the scope of the present invention is not limited by the dose in any way.

The term “individual” used herein refers to a subject in need of treatment, and more specifically, a mammal such as a human or a non-human primate, a mouse, a rat, a dog, a cat, a horse, or a cow.

Hereinafter, preferred preparation examples and examples are presented to better understand the present invention. However, the following examples are provided to more easily understand the present invention, and the content of the present invention is not limited by the following preparation examples.

### <Preparation Example 1> Ethyl 3, 4-Dihydroxycyclopentane-1-Carboxylate (4)

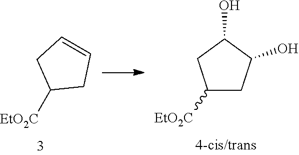 [SMILES: CCOC(=O)C1CC=CC1 ; CCOC(=O)C1C[C@@H](O)[C@@H](O)C1]

Compound 3 (2.5 g, 17.85 mmol) was dissolved in a solvent (28.5 mL) in which H₂O, THF, and t-BuOH were mixed in a volume ratio of 10:3:5, N-methylmorpholine N-oxide (NMO; 4.8 g, 35.7 mmol) was added and then OsO₄ (2 mL, 0.035 mmol) was added, and the resulting mixture was stirred at room temperature for 2 hours. After confirming the termination of the reaction, the resulting mixture was extracted with ethyl acetate (EA), and the organic layer was washed with water and brine. After removing water with anhydrous magnesium sulfate (MgSO₄) and concentrating the solvent, Compound 4 (2.9 g, 93%) was obtained.

4-trans (major 2.18 g)¹H NMR (400 MHz, DMSO) δ 4.46 (d, J=4.4 Hz, 2H), 4.03 (q, J=7.1 Hz, 3H), 3.90-3.84 (m, 2H), 2.94 (tt, J=9.6, 6.7 Hz, 1H), 1.86-1.74 (m, 4H), 1.16 (t, J=7.1, 0.6 Hz, 3H).

4-cis (minor 0.73 g)¹H NMR (400 MHz, DMSO) δ 4.37 (d, J=4.5 Hz, 2H), 4.03 (q, J=7.1 Hz, 2H), 3.78-3.72 (m, 2H), 2.72-2.60 (tt, 1H), 1.98-1.90 (m, 4H), 1.24 (t, J=8.4, 5.8 Hz, 3H). HRMS (ESI⁺) calculated for C₈H₁₅O₄ [M+H]+: 175.0965, found 175.2584.

### <Preparation Example 2> Ethyl 3, 4-bis((methylsulfonyl)oxy)cyclopentane-1-carboxylate (4′)

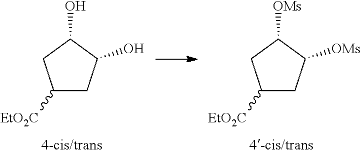 [SMILES: CCOC(=O)C1C[C@@H](O)[C@@H](O)C1 ; CCOC(=O)C1C[C@H](OS(C)(=O)=O)[C@H](OS(C)(=O)=O)C1]

Compound 4 (2.9 g, 16.9 mmol), which had been obtained in the previous experiment, was dissolved in MC (85 mL), TEA (5.1 mL, 37.1 mmol) was added at 0° C., and then the resulting mixture was stirred for 10 minutes. Afterward, methanesulfonic acid (MsCl; 2.85 mL, 37.1 mmol) was added and then stirring was performed at room temperature for 2 hours. After confirming the termination of the reaction, the resulting product was extracted with ethyl acetate (EA), and the organic layer was washed with water and brine. After removing water with anhydrous magnesium sulfate (MgSO₄) and concentrating the solvent, Compound 4′(5.58 g) was obtained.

4′-trans (major 4.19 g)¹H NMR (400 MHz, CDCl₃) δ 5.10 (p, J=3.7 Hz, 2H), 4.21-4.11 (q, 2H), 3.29-3.14 (m, 1H), 3.10 (s, 6H), 2.41-2.28 (m, 4H), 1.29-1.23 (t, 3H).

4′-cis (minor 1.39 g)¹H NMR (400 MHz, CDCl₃) δ 5.03-4.97 (m, 2H), 4.11-3.99 (q, 2H), 3.67 (s, 6H), 2.93-2.86 (m, 1H), 2.51-2.42 (m, 4H), 1.33 (t, J=7.1 Hz, 3H).

### <Preparation Example 3> Ethyl 3,4-diazidocyclopentane-1-carboxylate (5)

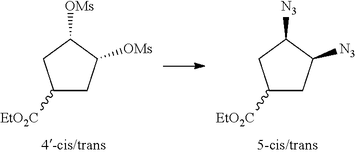 [SMILES: CCOC(=O)C1C[C@H](OS(C)(=O)=O)[C@H](OS(C)(=O)=O)C1 ; CCOC(=O)C1C[C@H](N=[N+]=[N-])[C@H](N=[N+]=[N-])C1]

Compound 4 (2.9 g, 16.9 mmol), which had been obtained in the previous experiment, was dissolved in DMF (85 mL), sodium azide (NaN₃; 5.5 g, 85 mmol) was added, and then the resulting mixture was stirred at 80° C. for 24 hours. After confirming the termination of the reaction, the resulting product was extracted with ethyl acetate (EA), and the organic layer was washed with water and brine. After removing water with anhydrous magnesium sulfate (MgSO₄) and concentrating the solvent, the resulting solution was purified by column chromatography using HEX and EA (HEX:EA=10:1) as mobile phase, thereby obtaining Compound 5.

5-trans (minor 0.62 g)¹H NMR (400 MHz, CDCl₃) δ 4.14 (q, J=7.1 Hz, 2H), 4.00 (p, J=4.2 Hz, 2H), 3.10 (tt, J=9.8, 6.2 Hz, 1H), 2.22-2.07 (m, 8H), 1.26 (t, J=7.1 Hz, 3H).

5-cis (major 1.85 g) ¹H NMR (400 MHz, CDCl₃) δ 4.18 (q, J=7.1 Hz, 2H), 3.89-3.83 (m, 2H), 2.87 (p, J=8.4 Hz, 1H), 2.27-2.20 (m, 4H), 1.27 (t, J=7.1 Hz, 3H).

### <Preparation Example 4> Ethyl 3,4-diaminocyclopentane-1-carboxylate (6)

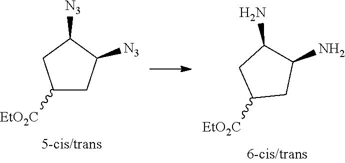 [SMILES: CCOC(=O)C1C[C@H](N=[N+]=[N-])[C@H](N=[N+]=[N-])C1 ; CCOC(=O)C1C[C@@H](N)[C@@H](N)C1]

Compound 5 (710 mg, 3.17 mmol), which had been obtained in the previous experiment, was dissolved in MeOH (12 mL), and then 20% Pd(OH)₂ on carbon (220 mg, 0.317 mmol) was added. The reaction vessel was substituted with hydrogen gas and stirred at room temperature for 1 hour. After confirming the termination of the reaction, Pd(OH)₂ was removed by filtration using Celite with ethyl acetate as a solvent, and the solvent was concentrated, thereby obtaining Compound 6 (533 mg, 97%).

6-trans (minor 0.14 g)¹H NMR (400 MHz, CDCl₃) δ 4.15-4.09 (q, 2H), 3.47-3.41 (m, 2H), 3.12 (td, J=9.6, 4.5 Hz, 1H), 2.20-2.11 (m, 2H), 1.96-1.87 (m, 2H), 1.27-1.22 (t, 3H).

6-cis (major 0.40 g)¹H NMR (400 MHz, CDCl₃) δ 4.14 (q, J=11.0, 4.0 Hz, 2H), 3.28 (dd, J=7.7, 3.2 Hz, 2H), 2.89-2.72 (m, 1H), 2.20 (dd, J=9.1, 6.2 Hz, 2H), 1.89-1.77 (m, 2H), 1.28-1.23 (t, 3H).

### <Preparation Example 5> Ethyl 2-aryl-1, 3a, 4, 5, 6, 6a-hexahydrocyclopenta [d]imidazol-5-carboxylate (8a-e)

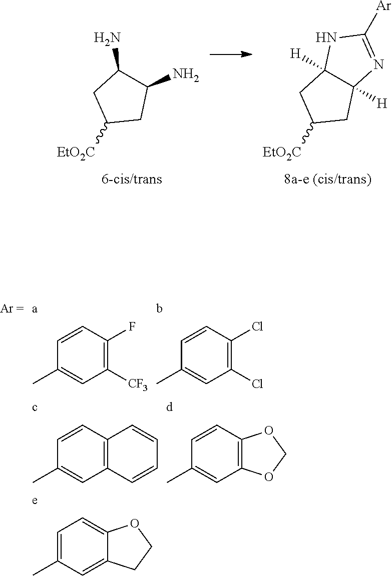

Compound 6 (1.119 g, 6.50 mmol), which had been obtained in the previous experiment, was dissolved in EtOH, and then ethyl 2-naphthimidate was added. The resulting mixture was stirred at 80° C. for 12 hours. After confirming the termination of the reaction, the resulting product was extracted with ethyl acetate (EA), and the organic layer was washed with water and brine. After removing water with anhydrous magnesium sulfate (MgSO₄) and concentrating the solvent, the resulting solution was purified by column chromatography using MC, MeOH, and NH₄OH (MC:MeOH:NH₄OH=100:10:1) as a mobile phase, thereby obtaining Compound 8a (1.91 g, 85%).

8a (1.9062 g, 85%). ¹H NMR (400 MHz, DMSO-d6) δ 8.19-8.12 (m, 2H), 7.63-7.56 (m, 1H), 4.50 (d, J=1.7 Hz, 2H), 4.04 (td, J=7.1, 1.7 Hz, 2H), 3.41-3.23 (m, 1H), 2.68-2.57 (m, 1H), 1.99-1.84 (m, 4H), 1.19-1.13 (m, 3H). HRMS (ESI⁺) calculated for C₁₆H₁₇F₄N₂O₂ [M+H]+: 345.1221, found 345.2313.

8b (1005 mg, 53%)¹H NMR (400 MHz, DMSO) δ 8.00 (d, J=1.9 Hz, 1H), 7.77 (dd, J=8.4, 1.9 Hz, 1H), 7.71 (d, J=8.4 Hz, 1H), 4.49 (s, 2H), 4.04 (q, J=7.1 Hz, 2H), 3.59 (s, 1H), 2.67-2.56 (m, 1H), 1.99-1.84 (m, 4H), 1.16 (t, J=7.1 Hz, 3H); MS(ESI⁺) calculated for C₁₅H₁₇C₁₂N₂O₂⁺ [M+H]+: 327.0662, found 327.0825.

8c (2100 mg, 69%). ¹H NMR (400 MHz, DMSO-D6) δ 8.32 (s, 1H), 7.98-7.91 (m, 4H), 7.61-7.54 (m, 2H), 7.40 (d, J=47.8 Hz, 1H), 4.53 (s, 2H), 4.05 (q, J=7.1 Hz, 2H), 2.64 (dt, J=17.9, 5.9 Hz, 1H), 2.04-1.96 (m, 2H), 1.91 (td, J=12.5, 6.5 Hz, 2H), 1.19-1.13 (t, 3H). ¹H NMR (400 MHz, DMSO) δ 8.32 (s, 1H), 7.97-7.92 (m, 4H), 7.58-7.55 (m, 2H), 4.35 (s, 2H), 3.90 (q, J=7.1 Hz, 2H), 2.73 (t, J=7.4 Hz, 1H), 2.27 (dt, J=12.9, 7.3 Hz, 2H), 1.95-1.87 (m, 2H), 1.05 (t, J=7.1 Hz, 3H). HRMS (ESI⁺) calculated for C₁₉H₂₀N₂O₂ [M+H]+: 308.3810, found 309.2216.

8d (601 mg, 47%). ¹H NMR (400 MHz, Acetone) δ 7.45-7.40 (m, 2H), 6.85 (dd, J=8.5, 3.6 Hz, 1H), 6.04 (d, J=1.7 Hz, 2H), 4.56 (dd, J=4.4, 2.2 Hz, 1H), 4.38 (dd, J=6.1, 2.9 Hz, 1H), 4.07 (q, J=8.5, 5.7 Hz, 2H), 2.77-2.71 (m, 1H), 2.33-2.23 (m, 2H), 1.99-1.91 (m, 2H), 1.19 (t, J=7.6, 6.7 Hz, 3H); ¹H NMR (400 MHz, Acetone) δ 7.45-7.40 (m, 2H), 6.85 (dd, J=8.5, 3.6 Hz, 1H), 6.04 (d, J=1.7 Hz, 2H), 4.56 (dd, J=4.4, 2.2 Hz, 1H), 4.38 (dd, J=6.1, 2.9 Hz, 1H), 4.00-3.94 (q, 2H), 2.77-2.71 (m, 1H), 2.33-2.23 (m, 2H), 1.99-1.91 (m, 2H), 1.13 (t, J=7.1 Hz, 3H); HRMS (ESI⁺) calculated for C₁₆H₁₈N₂O₄ [M+H]+: 302.3300, found 303.2079.

8e (590 mg, 46%)¹H NMR (400 MHz, DMSO-d6) δ 10.80 (s, 1H), 7.94 (s, 1H), 7.86 (dd, J=8.5, 2.0 Hz, 1H), 6.99 (d, J=8.5 Hz, 1H), 4.68 (dd, J=10.5, 7.1 Hz, 4H), 3.25 (t, J=8.7 Hz, 2H), 3.07 (m, J=4.8 Hz, 1H), 2.31 (dd, J=14.0, 3.0 Hz, 2H), 2.22-2.14 (m, 2H), 1.20 (t, 3H); ¹H NMR (400 MHz, DMSO-d6) δ 10.80 (s, 1H), 7.94 (s, 1H), 7.86 (dd, J=8.5, 2.0 Hz, 1H), 6.99 (d, J=8.5 Hz, 1H), 4.68 (dd, J=10.5, 7.1 Hz, 4H), 4.08 (q, J=7.1 Hz, 1H), 3.25 (t, J=8.7 Hz, 2H), 3.07 (d, J=4.8 Hz, 1H), 2.31 (dd, J=14.0, 3.0 Hz, 2H), 2.22-2.14 (m, 2H), 1.03 (t, J=7.1 Hz, 3H). HRMS (ESI⁺) calculated for C₁₇H₂₁N₂O₃ [M+H]+: 301.1547, found 301.2634.

### <Preparation Example 6> Ethyl 2-aryl-1, 4, 5, 6-tetrahydrocyclopenta[d]imidazol-5-carboxylate (9a-e)

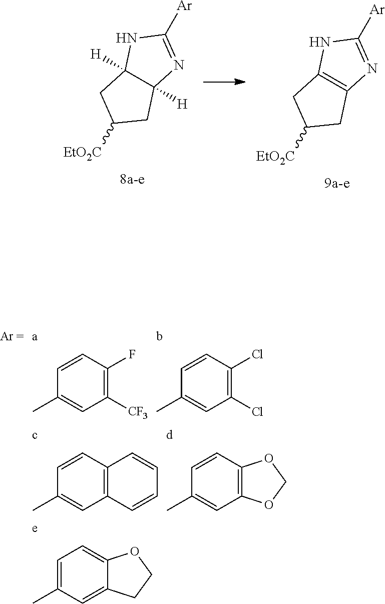

Oxalyl chloride (519 μL, 6.05 mmol) was dissolved in MC (15 mL), and stirred at −78° C. for approximately 10 minutes. Dimethyl sulfoxide (DMSO; 780 μL, 11 mmol) was dissolved in MC (15 mL), and then added to the oxalyl chloride being stirred, followed by stirring for 15 minutes. Compound 8a (1.89 g, 5.5 mmol), which had been obtained in the previous experiment, was dissolved in MC (25 mL) and added dropwise to a reaction vessel while stirring, and then further stirred for 30 minutes. Afterward, triethyl amine (TEA; 3.2 mL, 27.5 mmol) was added dropwise. After stirring for 10 minutes, the resulting solution was brought to room temperature to proceed with the reaction. After confirming the completion of the reaction, the resulting product was extracted with ethyl acetate (EA), and the organic layer was washed with water and brine. After removing water with anhydrous magnesium sulfate (MgSO₄) and concentrating the solvent, the resulting solution was purified by column chromatography using HEX and EA (HEX:EA=1:2) as mobile phase, thereby obtaining Compound 9.

9a (1.243 g, 66%). ¹H NMR (400 MHz, DMSO-d6) δ 12.57 (s, 1H), 8.21 (dd, J=6.9, 2.0 Hz, 1H), 8.19-8.14 (m, 1H), 7.56 (dd, J=10.3, 9.1 Hz, 1H), 4.13 (q, J=7.1 Hz, 2H), 3.79 (tt, J=9.1, 6.1 Hz, 1H), 3.09-2.85 (m, 3H), 2.78 (dd, J=14.5, 5.6 Hz, 1H), 1.22 (t, J=7.1 Hz, 3H). HRMS (ESI⁺) calculated for C₁₆H₁₈F₄N₂O₂ [M+H]+: 343.2148, found 343.2148.

9b (209 mg, 22%)¹H NMR (400 MHz, DMSO) δ 12.53 (s, 1H), 8.06 (d, J=2.0 Hz, 1H), 7.82 (dd, J=8.5, 2.1 Hz, 1H), 7.67 (d, J=8.5 Hz, 1H), 4.13 (q, J=7.1 Hz, 2H), 3.79 (ddd, J=9.2, 7.7, 4.6 Hz, 1H), 3.08-2.85 (m, 4H), 1.22 (t, J=7.1 Hz, 3H). MS(ESI⁺) calculated for C₁₅H₁₅C₁₂N₂O₂⁺ [M+H]+: 325.0505, found 325.4621.

9c (1030 mg, 41%). ¹H NMR (400 MHz, DMSO) δ 12.50 (s, 1H), 8.35 (s, 1H), 8.04 (dd, J=8.6, 1.7 Hz, 1H), 7.94 (s, 1H), 7.89 (d, J=8.8 Hz, 2H), 7.55-7.47 (m, 2H), 4.14 (q, J=6.9 Hz, 2H), 3.81 (ddd, J=9.1, 7.6, 4.6 Hz, 1H), 3.00 (dd, J=14.4, 9.2 Hz, 2H), 2.90 (dd, J=13.9, 6.1 Hz, 2H), 1.23 (t, J=7.1 Hz, 3H); ¹³C NMR (101 MHz, DMSO-D6) δ 175.03 (s), 174.58 (s), 149.25 (s), 133.06 (s), 132.36 (s), 128.99 (s), 128.20 (s), 127.89 (s), 127.66 (s), 126.59 (s), 125.94 (s), 122.97 (s), 122.36 (s), 60.31 (s), 51.80 (s), 45.89 (d, J=11.3 Hz), 27.40 (s), 14.12 (s); HRMS (ESI⁺) calculated for C₁₉H₁₈N₂O₂ [M+H]+: 306.3650, found 307.2050.

9d (717 mg, 58%). ¹H NMR (400 MHz, Acetone) δ 7.42 (dd, J=7.4, 1.6 Hz, 2H), 6.89-6.85 (m, 1H), 6.02 (s, 2H), 4.16 (q, J=7.1 Hz, 2H), 3.74 (dd, J=15.2, 8.0 Hz, 1H), 3.01-2.83 (m, 4H), 1.25 (t, J=7.1 Hz, 3H). ¹³C NMR (101 MHz, DMSO-D6) δ 174.60 (s), 149.13 (s), 147.59 (s), 146.82 (s), 142.62 (s), 137.14 (s), 125.94 (s), 118.06 (s), 108.50 (s), 104.79 (s), 101.11 (s), 60.28 (s), 45.90 (s), 27.60 (s), 27.14 (s), 14.12 (s); HRMS (ESI⁺) calculated for C₁₆H₁₆N₂O₄ [M+H]+: 300.3140, found 301.1913.

9e (991 mg, 83%)¹H NMR (400 MHz, Acetone) 67.78 (d, J=1.3 Hz, 1H), 7.68-7.64 (m, 1H), 6.73 (d, J=8.4 Hz, 1H), 4.57 (t, J=11.5, 5.9 Hz, 2H), 4.16 (q, J=7.1 Hz, 2H), 3.75 (dt, J=15.3, 7.6 Hz, 1H), 3.22 (t, J=8.7 Hz, 2H), 2.90 (d, J=44.8 Hz, 4H), 1.25 (t, J=7.1 Hz, 3H). HRMS (ESI⁺) calculated for C₁₇H₁₉N₂O₃ [M+H]+: 299.1390, found 299.2108.

### <Preparation Example 7> Ethyl 1-(2-(methylthio)pyrimidin-4-yl)-2-aryl-1,4,5,6-tetrahydrocyclopenta[d] imidazol-5-carboxylate (10a-e)

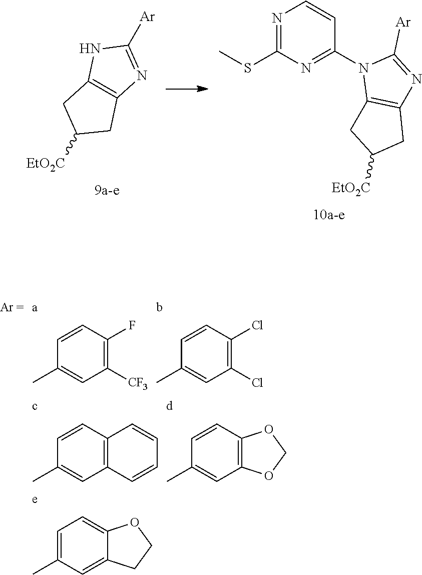

Compound 9a (268 mg, 0.78 mmol), which had been obtained in the previous experiment, was dissolved in anhydrous DMF (3.9 mL, 0.2M), and CsCO₃ (263 mg, 0.81 mmol) was added. 4-Chloro-2-(methylthio)pyrimidine (91 μL, 0.78 mmol) was added, and then stirred in a reactor microwave (Biotage) for 1 hour at 80° C. After completing the reaction, an organic layer was extracted using ethyl acetate (EtOAc), and washed with water and brine. After removing water with anhydrous magnesium sulfate (MgSO₄) and evaporating the solvent, separation and purification were performed through column chromatography (MC:MeOH=40:1), thereby obtaining Compound 10a (179 mg, 49%).

¹H NMR (400 MHz, DMSO) δ 8.65 (d, J=5.4 Hz, 1H), 7.81 (dd, J=6.8, 1.7 Hz, 1H), 7.72 (dd, J=5.5, 3.1 Hz, 1H), 7.56-7.48 (m, 1H), 7.07 (d, J=5.4 Hz, 1H), 4.14 (q, J=7.1 Hz, 2H), 3.86 (tt, J=9.2, 6.1 Hz, 1H), 3.24 (ddd, J=21.7, 15.5, 7.4 Hz, 2H), 3.00 (dd, J=14.8, 9.5 Hz, 1H), 2.89 (dd, J=15.0, 5.9 Hz, 1H), 2.16 (s, 3H), 1.22 (t, J=7.1 Hz, 3H); HIRMS (ESI⁺) calculated for C₂₁H₁₉F₄N₄O₂S [M+H]+: 467.1159, found 467.3361.

10b (135 mg, 49%)¹H NMR (400 MHz, DMSO) δ 8.66 (d, J=5.4 Hz, 1H), 7.71 (d, J=2.0 Hz, 1H), 7.64 (d, J=8.4 Hz, 1H), 7.33 (dd, J=8.4, 2.1 Hz, 1H), 7.04 (d, J=5.4 Hz, 1H), 4.14 (q, J=7.1 Hz, 2H), 3.86 (tt, J=9.2, 6.0 Hz, 1H), 3.25 (d, J=9.9 Hz, 1H), 3.18 (dd, J=15.8, 5.6 Hz, 1H), 3.00 (dd, J=15.5, 8.9 Hz, 1H), 2.89 (dd, J=15.0, 5.9 Hz, 1H), 2.22 (s, 3H), 1.22 (s, 3H); MS(ESI⁺) calculated for C₂₀H₁₉Cl₂N₄O₂S⁺ [M+H]+: 449.0600, found 449.7283.

10c (717 mg, 59%). ¹H NMR (400 MHz, Acetone) δ 8.48 (d, J=5.4 Hz, 1H), 8.06 (d, J=0.9 Hz, 1H), 7.95-7.89 (m, 3H), 7.58-7.53 (m, 3H), 6.81 (d, J=5.4 Hz, 1H), 4.19 (q, J=7.1, 4.8 Hz, 2H), 3.87 (tt, J=8.9, 6.4 Hz, 1H), 3.40-3.32 (m, 2H), 3.07-2.99 (m, 2H), 2.28 (s, 3H), 1.30-1.26 (t, 3H). ¹³C NMR (101 MHz, DMSO-D6) δ 174.21 (s), 172.18 (s), 159.60 (s), 156.21 (s), 149.85 (s), 144.82 (s), 133.63 (s), 132.61 (d, J=4.0 Hz), 128.74 (s), 128.20 (s), 127.77 (s), 127.55 (s), 126.83 (s), 126.57 (s), 126.03 (s), 109.21 (s), 59.72 (s), 45.16 (s), 28.53 (s), 28.13 (s), 14.05 (d, J=4.2 Hz). IRMS (ESI⁺) calculated for C₂₄H₂₂N₄O₂S [M+H]+: 430.5260, found 431.7963.

10d (713 mg, 72%). ¹H NMR (400 MHz, MeOD) δ 8.46 (d, J=5.5 Hz, 1H), 6.92-6.87 (m, 3H), 6.73 (d, J=5.5 Hz, 1H), 6.03 (s, 2H), 4.22 (q, J=7.1 Hz, 2H), 3.86 (ddd, J=12.8, 7.5, 4.3 Hz, 1H), 3.32-3.29 (m, 2H), 3.08-2.99 (m, 2H), 1.33-1.29 (t, 3H). 13C NMR (101 MHz, DMSO-D6) δ 174.27 (s), 172.15 (s), 159.51 (s), 156.24 (s), 149.59 (s), 147.84 (s), 147.29 (s), 144.21 (s), 133.07 (s), 125.11 (s), 122.96 (s), 109.31 (s), 108.86 (s), 108.33 (s), 101.43 (s), 60.43 (s), 45.07 (s), 28.57 (s), 28.13 (s), 14.09 (s), 13.26 (s). HIRMS (ESI⁺) calculated for C₂₁H₂₀N₄O₄S [M+H]+: 424.4750, found 425.7469.

10e (12 mg, 17%)¹H NMR (400 MHz, DMSO-d6) δ 8.54 (d, J=5.5 Hz, 1H), 7.29 (d, J=1.0 Hz, 1H), 7.07 (dd, J=8.3, 1.8 Hz, 1H), 6.74 (dd, J=6.8, 5.4 Hz, 2H), 4.56 (t, J=8.7 Hz, 2H), 4.14 (q, J=7.1 Hz, 2H), 3.81 (tt, J=9.2, 6.0 Hz, 1H), 3.29-3.21 (m, 1H), 3.16 (dd, J=11.7, 6.0 Hz, 3H), 2.96 (dd, J=14.7, 9.4 Hz, 1H), 2.86 (dd, J=14.9, 5.8 Hz, 1H), 2.34 (s, 3H), 1.22 (t, J=7.1 Hz, 3H). HRMS (ESI⁺) calculated for C₂₂H₂₃N₄O₃S [M+H]+: 423.1485, found 423.2263.

### <Preparation Example 8> Ethyl 1-(2-(methylthio)pyrimidin-4-yl)-2-aryl-1,4,5,6-tetrahydrocyclopenta[d]imidazol-5-carboxylate (11a-e)

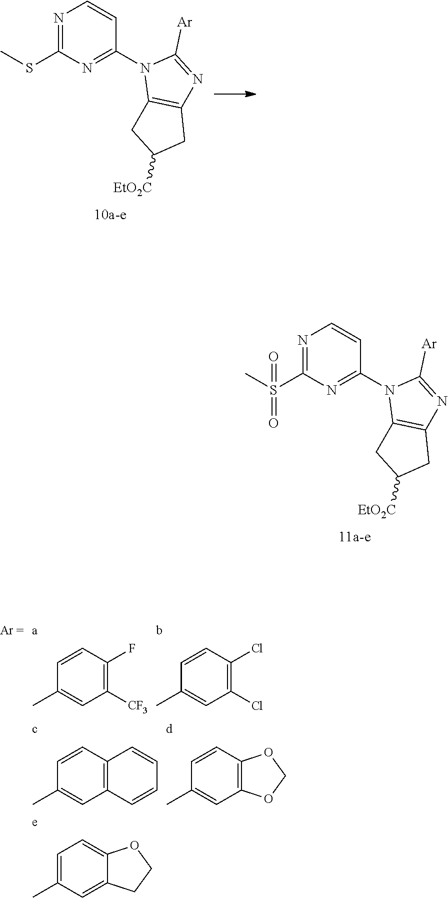

Compound 10a (364 mg, 0.78 mmol), which had been obtained in the previous experiment, and potassium peroxomonosulfate were mixed in a solvent in which methanol and water (1:1) were mixed (15.6 mL), and the resulting mixture was stirred at room temperature for 1 hour. After the starting material completely disappeared in TLC, the methanol was concentrated in a vacuum. The concentrated mixture was diluted by adding water, stirred until a solid product was separated, and then filtrated, thereby obtaining the solid product (Compound 11a; 136.8 mg, 0.27 mmol).

11a (136.8 mg, 35%). ¹H NMR (400 MHz, DMSO-d6) δ 8.38 (dd, J=14.8, 5.9 Hz, 1H), 7.79-7.62 (m, 2H), 7.58-7.47 (m, 1H), 6.37 (dd, J=42.0, 36.8 Hz, 1H), 4.19-4.06 (m, 2H), 3.88-3.79 (m, 1H), 3.32 (s, 3H), 3.23-3.16 (m, 1H), 3.14-3.03 (m, 1H), 2.98 (dd, J=14.8, 8.9 Hz, 1H), 2.87 (dd, J=16.1, 6.2 Hz, 1H), 1.21 (dd, J=5.3, 3.0 Hz, 3H). HRMS (ESI⁺) calculated for C₂₁H₁₉F₄N₄O₄S [M+H]+: 499.1058, found 499.3828.

11b (35 mg, 38%)¹H NMR (400 MHz, DMSO) δ 8.66 (d, J=5.4 Hz, 1H), 7.71 (d, J=2.0 Hz, 1H), 7.64 (d, J=8.4 Hz, 1H), 7.33 (dd, J=8.4, 2.1 Hz, 1H), 7.04 (d, J=5.4 Hz, 1H), 4.14 (q, J=7.1 Hz, 2H), 3.86 (tt, J=9.2, 6.0 Hz, 1H), 3.22 (s, 3H), 3.25 (d, J=9.9 Hz, 1H), 3.18 (dd, J=15.8, 5.6 Hz, 1H), 3.00 (dd, J=15.5, 8.9 Hz, 1H), 2.89 (dd, J=15.0, 5.9 Hz, 1H), 2.22 (s, 3H); MS(ESI⁺) calculated for C₂₀H₁₉Cl₂N₄O₄SV[M+H]+: 481.0499, found 481.8831.

11c (43 mg, 56%). 1H NMR (400 MHz, DMSO-D6) δ 8.23 (d, J=5.3 Hz, 1H), 8.00 (s, 1H), 7.90 (d, J=8.6 Hz, 3H), 7.60-7.50 (m, 3H), 7.47-7.41 (m, 1H), 6.27 (s, 1H), 4.15 (q, J=7.0 Hz, 2H), 3.85 (dt, J=9.1, 7.7 Hz, 2H), 3.76 (s, 1H), 3.21 (dt, J=15.6, 8.8 Hz, 2H), 2.96 (ddd, J=20.1, 14.7, 7.4 Hz, 4H), 2.74-2.55 (m, 2H), 1.52 (d, J=49.5 Hz, 2H), 1.23 (t, J=7.1 Hz, 3H).

HRMS (ESI⁺) calculated for C₂₄H₂₂N₄O₄S [M+H]+: 462.5240, found 463.8073.

11d (250 mg, 82%). 1H NMR (400 MHz, DMSO-d6) δ 8.98 (d, J=5.6 Hz, 1H), 7.31 (d, J=5.6 Hz, 1H), 7.05 (dd, J=1.5, 0.5 Hz, 1H), 6.97-6.90 (m, 2H), 6.08 (s, 2H), 4.14 (q, J=7.1 Hz, 2H), 3.88-3.80 (m, 1H), 3.30 (dd, J=12.0, 2.5 Hz, 1H), 3.28 (s, 3H), 3.26-3.17 (m, 1H), 3.03-2.81 (m, 2H), 1.22 (t, J=7.1 Hz, 3H); HRMS (ESI⁺) calculated for C₂₁H₂₀N₄O₆S [M+H]+: 456.4730, found 457.2179.

11e (240 mg, 74%). ¹H NMR (400 MHz, DMSO-d6) δ 8.97 (d, J=5.6 Hz, 1H), 7.34 (s, 1H), 7.30 (d, J=5.6 Hz, 1H), 7.17 (dd, J=8.3, 2.0 Hz, 1H), 6.79 (d, J=8.3 Hz, 1H), 4.58 (t, J=8.8 Hz, 2H), 4.14 (q, J=7.1 Hz, 2H), 3.86-3.82 (m, 1H), 3.24 (s, 3H), 3.17 (s, 2H), 3.01 (d, J=9.1 Hz, 1H), 2.97 (d, J=10.3 Hz, 1H), 2.91-2.83 (m, 2H), 1.23 (d, J=7.1 Hz, 3H). HRMS (ESI⁺) calculated for C₂₂H₂₃N₄O₅S [M+H]+: 455.1384, found 455.2014.

### <Preparation Example 9> Ethyl 1-(2-(((S/R)-1-(tert-butoxycarbonyl)piperidine/pyrrolidine-3-yl)amino)pyrimidin-4-yl)-2-aryl-1,4,5,6-tetrahydrocyclopenta[d]imidazol-5-carboxylate (12a-e, 13a-e, 14a-e, 15a-e)

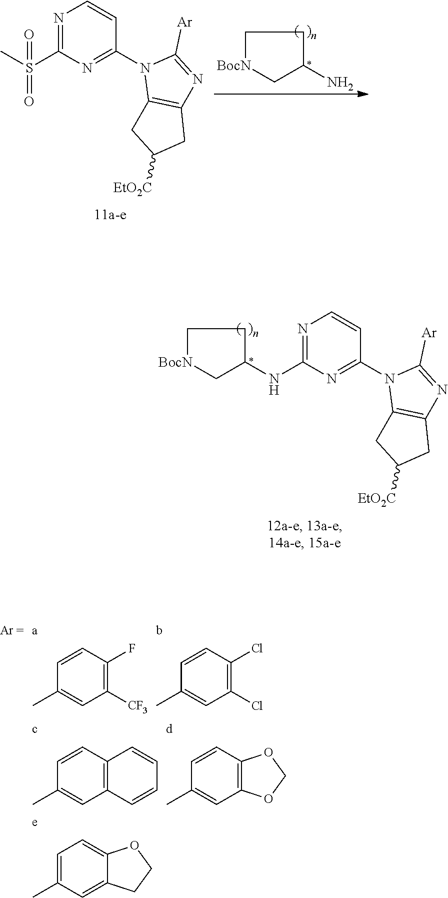

Compound 11 that had been obtained in the previous reaction was dissolved in THF (1.5 mL), tert-butyl 3-aminopyrrolidine-1-carboxylate (*=S, n=108.5 μL, 0.60 mmol) was added, and then the resulting mixture was stirred at 60° C. for 22 hours. After confirming the termination of the reaction, the resulting product was extracted with ethyl acetate (EtOAc), and the organic layer was washed with water and brine. After removing water with anhydrous magnesium sulfate (MgSO₄) and concentrating the solvent, the resulting solution was purified by column chromatography using MC and MeOH (MC:MeOH=40:1) as mobile phase, thereby obtaining Compound 12a (25 mg, 40%).

12a (81 mg, 89%). ¹H NMR (400 MHz, DMSO-d6) δ 8.36 (d, J=5.0 Hz, 1H), 7.76 (t, J=23.6 Hz, 3H), 7.50 (t, J=9.2 Hz, 1H), 6.48 (s, 1H), 4.13 (q, J=7.1 Hz, 2H), 3.88-3.79 (m, 1H), 3.29-3.08 (m, 5H), 2.98 (dd, J=14.3, 9.6 Hz, 2H), 2.88 (dd, J=14.8, 5.5 Hz, 1H), 1.79 (d, J=7.7 Hz, 1H), 1.61 (dd, J=21.3, 13.5 Hz, 1H), 1.38 (s, 9H), 1.22 (t, J=7.0 Hz, 4H). HRMS (ESI⁺) calculated for C₃₀H₃₅F₄N₆O₄ [M+H]+: 619.2650, found 619.3580.

12b (21 mg, 85%)¹H NMR (400 MHz, DMSO) δ 8.35 (d, J=5.1 Hz, 1H), 7.80 (s, 1H), 7.69-7.57 (m, 2H), 7.31 (d, J=8.2 Hz, 1H), 6.45 (s, 1H), 4.13 (q, J=7.1 Hz, 2H), 3.87-3.76 (m, 1H), 3.34 (s, 1H), 3.25-2.81 (m, 8H), 1.85-1.75 (m, 1H), 1.57-1.49 (m, 1H), 1.39 (d, J=4.3 Hz, 9H), 1.23 (d, J=7.0 Hz, 3H). MS(ESI⁺) calculated for C₂₈H₃₃C₁₂N₆O₄⁺ [M+H]+: 587.1935, found 587.8881.

12c (22 mg, 52%). ¹H NMR (400 MHz, DMSO-D6) δ 8.24 (d, J=5.3 Hz, 1H), 8.00 (s, 1H), 7.93-7.87 (m, 3H), 7.71 (s, 1H), 7.56-7.49 (m, 2H), 7.44 (d, J=8.4 Hz, 1H), 6.39-6.11 (m, 1H), 4.15 (q, J=7.1 Hz, 2H), 3.91-3.80 (m, 1H), 3.76-3.51 (m, 1H), 3.30-3.09 (m, 4H), 3.09-2.72 (m, 4H), 1.90-1.48 (m, 2H), 1.38 (d, J=6.2 Hz, 9H), 1.23 (t, J=7.1 Hz, 3H). ¹³C NMR (101 MHz, DMSO-D6) δ 174.33 (s), 162.07 161.52 (m), 156.67 (s), 155.05 (s), 153.40 (s), 144.31 (s), 132.55 (d, J=4.9 Hz), 129.63 (s), 128.22 (s), 127.84 (d, J=59.2 Hz), 126.64 (d, J=17.5 Hz), 126.17 (s), 100.94 (s), 78.17 (s), 60.42 (s), 50.73 (s), 50.33 (s), 45.18 (s), 43.63 (s), 30.61 (s), 29.79 (s), 28.45 (s), 28.15 (s), 14.10 (s). HRMS (ESI⁺) calculated for C₃₂H₃₆N₆O₄ [M+H]+: 568.6780, found 569.9207.

12d (61 mg, 49%). 1H NMR (400 MHz, DMSO-D6) δ 8.26 (d, J=5.2 Hz, 1H), 7.77 (s, 1H), 6.96-6.87 (m, 2H), 6.83 (dd, J=8.1, 1.3 Hz, 1H), 6.19 (d, J=32.4 Hz, 1H), 6.05 (s, 2H), 4.13 (q, J=7.1 Hz, 2H), 3.98 (t, J=29.9 Hz, 1H), 3.86-3.72 (m, 1H), 3.53-3.37 (m, 2H), 3.26-3.08 (m, 4H), 3.00-2.78 (m, 2H), 2.09-1.72 (m, 2H), 1.39 (d, J=4.4 Hz, 9H), 1.21 (t, J=7.1 Hz, 3H). ³C NMR (101 MHz, DMSO-D6) δ 174.37 (s), 162.29 (s), 161.98 (s), 155.74 (s), 153.58 (s), 147.65 (s), 147.19 (s), 143.72 (s), 133.15 (s), 131.68 (s), 122.72 (s), 108.72 (s), 108.18 (s), 102.48 (s), 101.40 (s), 78.25 (s), 60.40 (s), 54.89 (s), 51.00 (s), 45.12 (s), 43.87 (s), 30.71 (s), 29.88 (s), 28.52 (s), 28.06 (d, J=19.3 Hz), 14.09 (s). HRMS (ESI⁺) calculated for C₃₂H₃₆N₆O₄ [M+H]+: 562.6270, found 563.9796.

12e (43 mg, 63%). ¹H NMR (400 MHz, DMSO) δ 8.27 (dd, J=11.8, 5.3 Hz, 1H), 7.79 (d, J=22.4 Hz, 1H), 7.29 (s, 1H), 7.05 (d, J=8.2 Hz, 1H), 6.77-6.72 (m, 1H), 6.25 (d, J=34.8 Hz, 1H), 4.54 (dd, J=8.5, 6.1 Hz, 2H), 4.15-4.10 (m, 2H), 3.80 (s, 2H), 3.46 (d, J=5.4 Hz, 1H), 3.28-3.08 (m, 6H), 2.99-2.90 (m, 1H), 2.84 (dd, J=14.5, 5.5 Hz, 1H), 1.99-1.90 (m, 1H), 1.75 (dt, J=12.4, 6.2 Hz, 1H), 1.48 (ddd, J=12.6, 5.9, 3.8 Hz, 1H), 1.21 (dd, J=7.0, 1.3 Hz, 3H), 0.81 (t, J=2.7 Hz, 1H), 0.77 (dd, J=4.9, 2.1 Hz, 2H), 0.71 (d, J=2.0 Hz, 2H); HRMS (ESI⁺) calculated for C₃₀H₃₇N₆O₅ [M+H]+: 561.2820, found 561.3150.

13a (79 mg, 87%). ¹H NMR (400 MHz, DMSO-d6) δ 8.36 (d, J=5.0 Hz, 1H), 7.76 (t, J=23.7 Hz, 3H), 7.50 (t, J=9.2 Hz, 1H), 6.48 (s, 1H), 4.13 (q, J=7.1 Hz, 2H), 3.83 (dd, J=13.4, 7.2 Hz, 1H), 3.28-3.06 (m, 5H), 2.99 (dd, J=14.4, 9.6 Hz, 2H), 2.88 (dd, J=14.9, 5.4 Hz, 1H), 1.79 (dd, J=6.6, 5.1 Hz, 1H), 1.62 (dd, J=17.4, 9.7 Hz, 1H), 1.38 (s, 9H), 1.21 (dd, J=13.7, 6.6 Hz, 4H). HRMS (ESI⁺) calculated for C₃₀H₃₅F₄N₆O₄ [M+H]+: 619.2650, found 619.4301.

13b (41 mg, 84%)¹H NMR (400 MHz, DMSO) δ 8.35 (d, J=5.2 Hz, 1H), 7.80 (s, 1H), 7.69-7.59 (m, 2H), 7.31 (d, J=8.3 Hz, 1H), 6.45 (s, 1H), 4.13 (d, J=7.1 Hz, 2H), 3.88-3.78 (m, 1H), 3.36 (s, 1H), 3.29-2.79 (m, 8H), 1.85-1.75 (m, 1H), 1.62-1.46 (m, 1H), 1.39 (d, J=4.7 Hz, 9H), 1.21 (d, J=7.0 Hz, 3H). MS(ESI⁺) calculated for C₂₈H₃₃Cl₂N₆O₄⁺ [M+H]+: 587.1935, found 587.9601.

13c (24 mg, 71%). 1H NMR (400 MHz, DMSO-D6) δ 8.24 (d, J=5.3 Hz, 1H), 8.00 (s, 1H), 7.93-7.91 (m, 1H), 7.89 (d, J=8.4 Hz, 2H), 7.70 (s, 1H), 7.56-7.50 (m, 2H), 7.45 (d, J=8.4 Hz, 1H), 6.38-6.13 (m, 1H), 4.21-4.05 (m, 4H), 3.90-3.80 (m, 1H), 3.74-3.52 (m, 1H), 3.28-3.18 (m, 4H), 3.08-2.96 (m, 2H), 2.90 (dt, J=33.7, 12.3 Hz, 2H), 1.72-1.47 (m, 2H), 1.38 (d, J=6.1 Hz, 9H), 1.23 (t, J=7.1 Hz, 3H); 13C NMR (101 MHz, DMSO-D6) δ 174.33 (s), 162.10-161.70 (m), 153.47 (d, J=11.0 Hz), 144.31 (s), 132.69-132.24 (m), 128.13 (s), 127.54 (s), 126.64 (d, J=18.2 Hz), 126.12 (s), 125.85 (s), 124.56 (s), 102.10 (s), 78.17 (s), 60.42 (s), 50.74 (s), 50.39 (s), 48.59 (s), 45.19 (s), 30.61 (s), 29.78 (s), 28.45 (s), 28.15 (s), 14.11 (s). HRMS (ESI⁺) calculated for C₃₂H₃₆N₆O₄ [M+H]+: 568.6780, found 569.5607.

13d (109 mg, 89%). ¹H NMR (400 MHz, DMSO-D6) δ 8.26 (d, J=5.2 Hz, 1H), 7.77 (s, 1H), 6.96-6.79 (m, 3H), 6.19 (d, J=32.4 Hz, 1H), 6.05 (s, 2H), 4.13 (q, J=7.1 Hz, 2H), 4.09-3.88 (m, 1H), 3.85-3.74 (m, 1H), 3.44 (ddd, J=23.8, 13.6, 6.2 Hz, 2H), 3.30-3.06 (m, 4H), 3.02-2.79 (m, 2H), 2.08-1.72 (m, 2H), 1.39 (d, J=4.4 Hz, 9H), 1.21 (t, J=7.1 Hz, 3H). ¹³C NMR (101 MHz, DMSO-D6) δ 174.34 (s), 162.12-161.87 (m), 153.53 (d, J=6.2 Hz), 147.65 (s), 147.17 (d, J=0.9 Hz), 143.72 (s), 122.70 (s), 108.71 (s), 108.16 (s), 101.40 (d, J=1.7 Hz), 78.22 (s), 60.39 (s), 54.87 (s), 50.99 (s), 45.12 (s), 44.11 (s), 29.90 (s), 28.53 (s), 28.11 (d, J=6.4 Hz), 14.07 (s). HRMS (ESI⁺) calculated for C₃₂H₃₆N₆O₄ [M+H]+: 562.6270, found 563.9435.

13e (45 mg, 66%). ¹H NMR (400 MHz, DMSO) δ 8.27 (dd, J=12.0, 5.2 Hz, 1H), 7.87-7.71 (m, 1H), 7.29 (s, 1H), 7.05 (d, J=8.2 Hz, 1H), 6.74 (dd, J=8.3, 1.8 Hz, 1H), 6.35-6.06 (m, 1H), 4.59-4.50 (m, 2H), 4.13 (dd, J=14.0, 7.0 Hz, 2H), 3.76 (d, J=26.0 Hz, 2H), 3.46 (d, J=5.4 Hz, 1H), 3.30-3.09 (m, 6H), 3.00-2.90 (m, 1H), 2.84 (dd, J=14.5, 5.1 Hz, 1H), 1.93 (dd, J=18.0, 8.6 Hz, 1H), 1.75 (dt, J=12.8, 6.3 Hz, 1H), 1.49 (tt, J=7.7, 4.8 Hz, 1H), 1.21 (t, J=7.1 Hz, 3H), 0.81 (t, J=2.6 Hz, 1H), 0.79-0.76 (m, 2H), 0.73-0.71 (m, 2H). HRMS (ESI⁺) calculated for C₃₀H₃₇N₆O₅ [M+H]+: 561.2820, found 561.2791.

14a (77.5 mg, 83%). ¹H NMR (400 MHz, DMSO-d6) δ 8.36 (d, J=4.8 Hz, 1H), 7.70 (dd, J=28.3, 6.9 Hz, 2H), 7.56-7.48 (m, 1H), 6.48 (s, 1H), 4.17-4.10 (m, 2H), 3.83 (ddd, J=15.4, 9.2, 6.4 Hz, 1H), 3.61 (d, J=10.3 Hz, 2H), 3.13 (dd, J=32.8, 23.1 Hz, 2H), 3.04-2.94 (m, 2H), 2.93-2.82 (m, 2H), 1.61 (s, 2H), 1.43-1.26 (m, 9H), 1.22 (td, J=7.1, 5.0 Hz, 5H), 1.19-1.13 (m, 1H), 0.88-0.78 (m, 1H). HRMS (ESI⁺) calculated for C₂₉H₃₃F₄N₆O₄ [M+H]+: 605.2494, found 605.2434.

14b (43 mg, 85%)¹H NMR (400 MHz, DMSO-d6) 8.34 (d, J=5.1 Hz, 1H), 7.60 (d, J=8.5 Hz, 2H), 7.53 (s, 1H), 7.30-7.25 (m, 1H), 6.44 (s, 1H), 4.13 (dd, J=9.5, 4.0 Hz, 2H), 3.81 (td, J=9.1, 4.8 Hz, 1H), 3.74-3.43 (m, 2H), 3.23-2.73 (m, 7H), 1.83-1.49 (m, 4H), 1.36 (dd, J=10.5, 6.2 Hz, 9H), 1.24-1.21 (m, 3H); MS(ESI⁺) calculated for C₂₉H₃₅Cl₂N₆O₄⁺ [M+H]+: 601.2091, found 601.9667.

14c (49 mg, 90%). ¹H NMR (400 MHz, MeOD) δ 8.20 (d, J=5.2 Hz, 1H), 8.00 (s, 1H), 7.90 (dd, J=12.6, 5.6 Hz, 3H), 7.59-7.52 (m, 2H), 7.44 (d, J=8.3 Hz, 1H), 6.35 (s, 1H), 4.28-4.20 (m, 2H), 3.93-3.87 (m, 1H), 3.54 (dd, J=80.1, 58.5 Hz, 3H), 3.28 (s, 1H), 3.07 (ddd, J=31.5, 20.3, 11.9 Hz, 4H), 1.52-1.35 (m, 9H), 1.34-1.30 (m, 6H), 0.91 (d, J=7.0 Hz, 2H); ¹³C NMR (101 MHz, DMSO-D6) δ 174.29 (s), 161.77 (s), 160.40 (d, J=5.3 Hz), 153.82 (d, J=15.0 Hz), 144.22 (s), 132.69-132.41 (m), 128.18 (d, J=4.2 Hz), 127.55 (s), 126.72 (s), 126.31 (d, J=39.9 Hz), 126.01-125.94 (m), 101.23 (s), 78.44 (s), 60.40 (s), 54.88 (s), 47.49 (s), 46.76 (s), 45.17 (s), 43.71 (s), 28.61-28.55 (m), 28.45 (s), 28.33-27.48 (m), 22.97 (s), 14.10 (s); HRMS (ESI⁺) calculated for C₃₂H₃₆N₆O₄ [M+H]+: 582.7050, found 583.9634.

14d (102.1 mg, 95%). ¹H NMR (400 MHz, DMSO-D6) δ 8.25 (d, J=5.2 Hz, 1H), 7.43 (d, J=52.5 Hz, 1H), 6.84 (dt, J=8.1, 4.8 Hz, 3H), 6.14 (dd, J=25.2, 19.6 Hz, 1H), 6.05 (s, 2H), 4.12 (qd, J=7.0, 3.5 Hz, 2H), 3.84-3.69 (m, 2H), 3.66-3.32 (m, 2H), 3.23-2.57 (m, 6H), 1.96-1.41 (m, 4H), 1.41-1.26 (m, 9H), 1.22 (dd, J=7.1, 4.0 Hz, 3H); ¹³C NMR (101 MHz, DMSO-D6) δ 174.32 (s), 161.80 (s), 160.32 (d, J=2.6 Hz), 153.82 (d, J=3.4 Hz), 147.61 (s), 147.16 (d, J=3.5 Hz), 143.63 (s), 133.00 (s), 122.72 (s), 108.72 (s), 108.19 (s), 101.41 (d, J=6.3 Hz), 78.47 (s), 60.38 (s), 47.49 (s), 47.00 (s), 45.10 (d, J=4.8 Hz), 28.50 (s), 28.49-27.14 (m), 22.57 (s), 14.09 (s). HRMS (ESI⁺) calculated for C₃₂H₃₆N₆O₄ [M+H]+: 576.6540, found 577.9603.

14e (30 mg, 43%). ¹H NMR (400 MHz, DMSO-d6) δ 8.22 (d, J=5.1 Hz, 1H), 7.26 (s, 1H), 7.04 (d, J=8.0 Hz, 1H), 6.74 (d, J=8.2 Hz, 1H), 6.10 (d, J=25.8 Hz, 1H), 4.55 (t, J=8.7 Hz, 2H), 4.17-4.09 (m, 2H), 3.78 (d, J=5.6 Hz, 3H), 3.20 (s, 1H), 3.16 (t, J=8.6 Hz, 2H), 3.07 (d, J=5.4 Hz, 1H), 3.02-2.76 (m, 4H), 1.73 (d, J=26.4 Hz, 2H), 1.37 (dd, J=13.5, 7.6 Hz, 9H), 1.24-1.20 (m, 5H), 1.13 (dd, J=14.9, 7.7 Hz, 1H), 0.87-0.78 (m, 1H). HRMS (ESI⁺) calculated for C₃₁H₃₉N₆O₅ [M+H]+: 575.2976, found 575.2137.

15a (64 mg, 69%). ¹H NMR (400 MHz, DMSO-d6) δ 8.35 (d, J=4.9 Hz, 1H), 7.78-7.62 (m, 2H), 7.58-7.43 (m, 2H), 6.41 (d, J=48.8 Hz, 1H), 4.17-4.09 (m, 2H), 3.85-3.79 (m, 1H), 3.72-3.53 (m, 2H), 3.12 (dd, J=32.3, 25.7 Hz, 2H), 3.05-2.93 (m, 2H), 2.93-2.81 (m, 2H), 1.69-1.56 (m, 2H), 1.40 (dt, J=25.1, 11.5 Hz, 11H), 1.24-1.20 (m, 4H). HRMS (ESI⁺) calculated for C₂₉H₃₃F₄N₆O₄ [M+H]+: 605.2494, found 605.2074.

15b (98 mg, 98%)¹H NMR (400 MHz, DMSO) δ 8.35 (d, J=5.2 Hz, 1H), 7.61 (d, J=8.4 Hz, 2H), 7.54 (s, 1H), 7.33-7.27 (m, 1H), 6.45 (s, 1H), 4.18-4.11 (m, 2H), 3.88-3.80 (m, 1H), 3.76-3.54 (m, 2H), 3.28-2.81 (m, 7H), 1.88-1.49 (m, 4H), 1.39 (d, J=5.7 Hz, 9H), 1.24 (dd, J=5.5, 1.4 Hz, 3H). MS (ESI⁺) calculated for C₂₉H₃₅Cl₂N₆O₄⁺ [M+H]+: 601.2091, Found 601.0306.

15c (29 mg, 61%). ¹H NMR (400 MHz, DMSO-D6) δ 8.22 (d, J=5.2 Hz, 1H), 8.00 (s, 1H), 7.93-7.86 (m, 3H), 7.53 (p, J=6.8 Hz, 2H), 7.44 (s, 2H), 6.25 (s, 1H), 4.14 (dt, J=7.1, 4.6 Hz, 2H), 3.84 (dd, J=8.8, 5.9 Hz, 1H), 3.67 (dd, J=29.5, 25.0 Hz, 1H), 3.56-3.35 (m, 2H), 3.21 (d, J=27.7 Hz, 4H), 3.01 (dt, J=12.7, 7.7 Hz, 1H), 2.94-2.86 (m, 1H), 2.84-2.53 (m, 1H), 1.95-1.47 (m, 2H), 1.46-1.28 (m, 9H), 1.23 (t, J=3.5 Hz, 3H), 1.03-0.74 (m, 1H). ¹³C NMR (101 MHz, DMSO-D6) δ 174.31 (s), 161.78 (s), 160.46 (d, J=4.6 Hz), 153.75 (d, J=5.8 Hz), 144.23 (s), 132.59 (d, J=2.4 Hz), 128.18 (s), 127.57 (s), 126.74 (s), 126.63-126.57 (m), 126.33 (d, J=40.9 Hz), 125.39 (s), 123.95 (s), 102.31 (s), 78.45 (s), 60.41 (s), 48.60 (s), 47.51 (s), 46.81 (s), 45.18 (s), 29.08-28.72 (m), 28.72-28.59 (m), 28.72-27.65 (m), 14.11 (s); HRMS (ESI⁺) calculated for C₃₂H₃₆N₆O₄ [M+H]+: 582.7050, found 583.9995.

15d (99 mg, 92%). ¹H NMR (400 MHz, DMSO-D6) δ 8.25 (d, J=5.2 Hz, 1H), 7.55-7.34 (m, 1H), 6.91 (d, J=8.0 Hz, 2H), 6.81 (dd, J=8.1, 1.6 Hz, 1H), 6.23-6.11 (m, 1H), 6.05 (s, 2H), 4.13 (dt, J=7.0, 5.2 Hz, 2H), 3.86-3.74 (m, 2H), 3.66 (d, J=2.9 Hz, 1H), 3.62-3.34 (m, 2H), 3.28-3.01 (m, 3H), 2.89 (ddd, J=20.3, 14.9, 7.5 Hz, 3H), 2.62 (dd, J=27.7, 19.4 Hz, 1H), 1.75 (d, J=13.1 Hz, 2H), 1.37 (dt, J=14.5, 10.6 Hz, 9H), 1.21 (dd, J=7.1, 3.1 Hz, 3H); ¹³C NMR (101 MHz, DMSO-D6) δ 174.32 (s), 161.81 (s), 160.31 (d, J=8.1 Hz), 153.86 (s), 153.62 (s), 147.61 (s), 147.18 (d, J=3.3 Hz), 143.62 (s), 137.61 (s), 122.72 (s), 108.73 (s), 108.20 (s), 101.36 (d, J=5.1 Hz), 78.50 (s), 60.39 (s), 47.51 (s), 46.96 (s), 45.11 (d, J=5.3 Hz), 28.33 (d, J=33.9 Hz), 27.88 (d, J=13.9 Hz), 23.55 (s), 14.10 (s); HRMS (ESI⁺) calculated for C₃₂H₃₆N₆O₄ [M+H]+: 576.6540, found 577.9503.

15e (28 mg, 40%). ¹H NMR (400 MHz, DMSO-d6) δ 8.22 (d, J=5.1 Hz, 1H), 7.26 (s, 1H), 7.04 (d, J=8.0 Hz, 1H), 6.74 (d, J=8.2 Hz, 1H), 6.13 (s, 1H), 4.56 (t, J=8.7 Hz, 2H), 4.18-4.08 (m, 2H), 3.78 (d, J=5.5 Hz, 3H), 3.14 (dd, J=24.8, 16.1 Hz, 4H), 2.88 (ddd, J=20.2, 14.7, 7.2 Hz, 4H), 1.74 (d, J=25.4 Hz, 2H), 1.38 (d, J=3.5 Hz, 9H), 1.25-1.19 (m, 5H), 1.18-1.06 (m, 1H), 0.88-0.82 (m, 1H); HRMS (ESI⁺) calculated for C₃₁H₃₉N₆O₅ [M+H]+: 575.2976, found 575.5378.

### <Example 1> Ethyl-1-(2-(((S/R)-1-(cyclopropanecarbonyl)piperidine/pyrrolidine-3-yl)amino)pyrimidin-4-yl)-2-aryl-1,4,5,6-tetrahydrocyclopenta[d]imidazol-5-carboxylates (16a-e, 17a-e, 18a-e, and 19a-e)

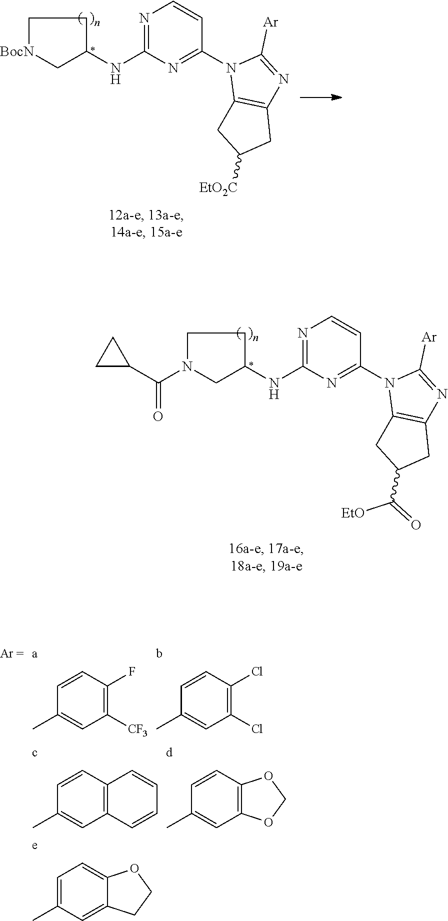

Compound 12a (25 mg, 0.043 mmol) was dissolved in MC (1.072 mL), 20% TFA (v/v) (268 μL) was added, and then the mixture was stirred for 30 minutes. After confirming the termination of the reaction, the resulting product was concentrated with MC three times. The resulting concentrate was dissolved in THF (1.15 mL, 0.1M), TEA (40 μL, 0.230 mmol) was added, and cyclopropanecarbonyl chloride (10.43 μL, 0.1.15 mmol) was slowly added and stirred. After confirming the termination of the reaction, the resulting product was extracted with ethyl acetate (EtOAc) and washed with water and brine. After drying water with anhydrous magnesium sulfate (MgSO₄), filtration and concentration were performed. Purification and separation were performed through column chromatography, thereby obtaining Compound 16a (44 mg, 66.8%).

16a (44 mg, 66.8%). ¹H NMR (400 MHz, DMSO-d6) δ 8.38 (s, 1H), 7.80 (t, J=37.9 Hz, 3H), 7.52 (s, 1H), 6.51 (d, J=17.9 Hz, 1H), 4.13 (d, J=6.3 Hz, 2H), 3.84 (s, 1H), 3.73 (s, 1H), 3.50 (d, J=52.3 Hz, 2H), 3.19 (d, J=24.7 Hz, 3H), 3.05-2.83 (m, 2H), 1.75 (d, J=29.3 Hz, 1H), 1.53 (s, 1H), 1.22 (s, 4H), 0.83 (d, J=9.4 Hz, 1H), 0.71 (s, 4H); HRMS (ESI⁺) calculated for C₂₈H₂₉F₄N₆O₃ [M+H]+: 573.2232, found 573.2693.

16b (22 mg, 58%)¹H NMR (400 MHz, DMSO) δ 8.37 (dd, J=13.9, 5.2 Hz, 1H), 7.92-7.77 (m, 1H), 7.66 (s, 1H), 7.63 (dd, J=8.4, 2.3 Hz, 1H), 7.32 (t, J=6.1 Hz, 1H), 6.50 (d, J=33.9 Hz, 1H), 4.13 (dt, J=10.4, 4.3 Hz, 2H), 3.83 (d, J=8.6 Hz, 1H), 3.75 (d, J=7.1 Hz, 1H), 3.58 (s, 1H), 3.46 (s, 1H), 3.23 (d, J=9.3 Hz, 2H), 3.15 (dd, J=16.2, 9.5 Hz, 2H), 2.98 (dd, J=15.5, 8.5 Hz, 1H), 2.87 (dd, J=14.8, 5.3 Hz, 1H), 1.87 (d, J=51.0 Hz, 2H), 1.76-1.67 (m, 1H), 1.24-1.19 (m, 3H), 0.71 (dd, J=8.2, 4.2 Hz, 4H); MS(ESI⁺) calculated for C₂₇H₂₉C₁₂N₆O₃⁺ [M+H]+: 555.1673, found 555.9500.

16c (13.9 mg, 55%). ¹H NMR (400 MHz, DMSO-D6) δ 8.36-8.23 (m, 1H), 8.00 (d, J=5.8 Hz, 1H), 7.90 (dd, J=10.3, 7.1 Hz, 3H), 7.74 (dd, J=26.5, 6.3 Hz, 1H), 7.58-7.41 (m, 3H), 6.31 (dd, J=72.1, 29.8 Hz, 1H), 4.20-4.11 (m, 2H), 3.86 (t, J=11.6 Hz, 1H), 3.64 (d, J=70.7 Hz, 1H), 3.53-3.33 (m, 2H), 3.30-3.04 (m, 4H), 2.95 (ddd, J=19.5, 14.1, 5.1 Hz, 2H), 1.97-1.34 (m, 3H), 1.23 (td, J=7.1, 1.9 Hz, 3H), 0.74-0.57 (m, 4H); ¹³C NMR (101 MHz, DMSO-D6) δ 174.33 (s), 170.32 (s), 162.17-161.71 (m), 157.04 (s), 144.31 (s), 132.53 (t, J=6.5 Hz), 131.26 (s), 128.13 (s), 127.47 (d, J=12.8 Hz), 126.64 (d, J=17.3 Hz), 126.08 (s), 122.93 (s), 102.19 (s), 60.44 (s), 51.95 (s), 51.12 (s), 49.06 (s), 45.20 (s), 30.80 (s), 28.16 (s), 20.75 (s), 14.11 (s), 11.57 (s), 6.91 (d, J=5.3 Hz). HRMS (ESI⁺) calculated for C₃₁H₃₂N₆O₃ [M+H]+: 536.6360, found 537.9465.

16d (42.4 mg, 26%). ¹H NMR (400 MHz, DMSO-D6) δ 8.29 (dd, J=9.9, 5.3 Hz, 1H), 7.81 (d, J=24.7 Hz, 1H), 6.98-6.87 (m, 2H), 6.83 (dd, J=8.1, 1.6 Hz, 1H), 6.31 (d, J=23.4 Hz, 1H), 6.09-6.00 (m, 2H), 4.13 (q, J=7.1 Hz, 2H), 4.06-3.75 (m, 2H), 3.74-3.55 (m, 1H), 3.54-3.34 (m, 2H), 3.30-3.03 (m, 3H), 2.90 (ddd, J=20.4, 14.8, 7.5 Hz, 2H), 2.14-1.57 (m, 3H), 1.25-1.17 (m, 3H), 0.71 (dd, J=9.3, 5.7 Hz, 4H); ¹³C NMR (101 MHz, DMSO-D6) δ 174.35 (s), 170.92 (s), 162.46 (s), 162.03 (d, J=13.6 Hz), 147.65 (s), 147.14 (d, J=5.3 Hz), 143.70 (s), 132.98 (s), 127.09 (s), 122.68 (s), 108.69 (s), 108.18 (s), 101.44 (d, J=8.5 Hz), 60.40 (s), 54.90 (s), 50.74 (s), 45.11 (s), 44.42 (s), 30.95 (s), 28.50 (s), 28.16 (s), 14.09 (s), 11.96 (s), 6.92 (d, J=4.3 Hz); HIRMS (ESI⁺) calculated for C₂₈H₃₀N₆O₅ [M+H]+: 530.5850, found 531.8253.

16e (9.1 mg, 33%). ¹H NMR (400 MHz, DMSO-d6) δ 8.27 (dd, J=11.8, 5.3 Hz, 1H), 7.79 (d, J=22.4 Hz, 1H), 7.29 (s, 1H), 7.05 (d, J=8.2 Hz, 1H), 6.77-6.72 (m, 1H), 6.25 (d, J=34.8 Hz, 1H), 4.54 (dd, J=8.5, 6.1 Hz, 2H), 4.15-4.10 (m, 2H), 3.80 (s, 2H), 3.46 (d, J=5.4 Hz, 1H), 3.28-3.08 (m, 6H), 2.99-2.90 (m, 1H), 2.84 (dd, J=14.5, 5.5 Hz, 1H), 1.99-1.90 (m, 1H), 1.75 (dt, J=12.4, 6.2 Hz, 1H), 1.48 (ddd, J=12.6, 5.9, 3.8 Hz, 1H), 1.21 (dd, J=7.0, 1.3 Hz, 3H), 0.81 (t, J=2.7 Hz, 1H), 0.77 (dd, J=4.9, 2.1 Hz, 2H), 0.71 (d, J=2.0 Hz, 2H); HIRMS (ESI⁺) calculated for C₂₉H₃₃N₆O₄ [M+H]+: 529.2558, found 529.3768.

17a (47.3 mg, 70%). ¹H NMR (400 MHz, DMSO-d6) δ 8.38 (dd, J=10.2, 5.2 Hz, 1H), 7.94-7.68 (m, 3H), 7.57-7.48 (m, 1H), 6.51 (d, J=19.8 Hz, 1H), 4.14 (q, J=7.1 Hz, 2H), 3.85 (s, 1H), 3.73 (s, 1H), 3.56 (s, 1H), 3.43 (d, J=7.2 Hz, 1H), 3.27-3.10 (m, 3H), 3.04-2.94 (m, 1H), 2.88 (dd, J=15.5, 4.8 Hz, 1H), 1.72 (s, 1H), 1.64-1.44 (m, 1H), 1.30-1.16 (m, 4H), 0.70 (dd, J=9.4, 5.4 Hz, 5H); IRMS (ESI⁺) calculated for C₂₈H₂₉F₄N₆O₄ [M+H]+: 573.2232, found 573.2333.

17b (27 mg, 78%)¹H NMR (400 MHz, DMSO) δ 8.37 (dd, J=14.1, 5.2 Hz, 1H), 7.93-7.79 (m, 1H), 7.66 (s, 1H), 7.63 (dd, J=8.4, 2.3 Hz, 1H), 7.35-7.29 (m, 1H), 6.50 (d, J=34.8 Hz, 1H), 4.13 (q, J=7.1 Hz, 2H), 3.84 (s, 1H), 3.74 (s, 1H), 3.66-3.40 (m, 2H), 3.29-2.83 (m, 6H), 2.01-1.91 (m, 1H), 1.75-1.70 (m, 1H), 1.55 (s, 1H), 1.22 (dd, J=7.1, 5.0 Hz, 3H), 0.71 (dd, J=8.2, 4.2 Hz, 4H); MS(ESI⁺) calculated for C₂₇H₂₉Cl₂N₆O₃ [M+H]+: 555.1673, Found 555.9140.

17c (13.8 mg, 76%). ¹H NMR (400 MHz, DMSO-D6) δ 8.34-8.23 (m, 1H), 8.00 (d, J=6.1 Hz, 1H), 7.90 (m, 3H), 7.75 (dd, J=26.5, 6.3 Hz, 1H), 7.57-7.49 (m, 2H), 7.45 (t, J=8.1 Hz, 1H), 6.40 (d, J=51.6 Hz, 1H), 4.19-4.11 (m, 2H), 3.87 (m, 1H), 3.57 (t, J=64.8 Hz, 2H), 3.28 (dd, J=18.4, 11.6 Hz, 4H), 3.22-3.14 (m, 1H), 3.13-2.96 (m, 2H), 2.91 (dd, J=14.6, 5.4 Hz, 1H), 1.60 (s, 2H), 1.23 (tt, J=7.0, 1.5 Hz, 3H), 0.70 (dd, J=20.0, 15.6 Hz, 4H); ¹³C NMR (101 MHz, DMSO-D6) δ 174.33 (s), 170.32 (s), 162.07-161.71 (m), 156.86 (s), 144.32 (s), 132.73-132.24 (m), 128.84 (s), 128.17 (d, J=8.4 Hz), 127.95-126.09 (m), 126.64 (d, J=16.4 Hz), 126.45 (t, J=32.4 Hz), 101.83 (s), 59.74 (s), 51.07 (s), 50.59 (s), 45.20 (s), 43.59 (s), 30.80 (s), 29.02 (s), 28.15 (s), 20.74 (s), 14.07 (s), 7.63 (s), 6.91 (s); HRMS (ESI⁺) calculated for C₃₁H₃₂N₆O₃ [M+H]+: 536.6360, found 537.6224.

17d (61.8 mg, 26%). ¹H NMR (400 MHz, DMSO-D6) δ 8.29 (dd, J=9.9, 5.3 Hz, 1H), 7.82 (d, J=25.5 Hz, 1H), 6.96-6.88 (m, 2H), 6.84 (dd, J=8.1, 1.6 Hz, 1H), 6.30 (d, J=22.7 Hz, 1H), 6.09-5.99 (m, 2H), 4.13 (q, J=7.1 Hz, 2H), 4.08-3.73 (m, 2H), 3.73-3.54 (m, 1H), 3.53-3.35 (m, 2H), 3.32-3.02 (m, 3H), 2.90 (ddd, J=20.5, 14.8, 7.5 Hz, 2H), 1.82 (dddd, J=53.0, 47.8, 42.4, 24.2 Hz, 3H), 1.25-1.17 (m, 3H), 0.75-0.66 (m, 4H); ¹³C NMR (101 MHz, DMSO-D6) δ 174.35 (s), 170.93 (s), 162.64 (s), 162.02 (d, J=10.4 Hz), 156.81 (s), 147.65 (s), 147.17 (s), 143.73 (s), 132.96 (s), 125.86 (s), 122.69 (s), 108.70 (s), 108.18 (s), 101.44 (d, J=8.5 Hz), 60.40 (s), 54.90 (s), 51.30 (s), 45.12 (s), 44.42 (s), 30.96 (s), 28.89 (d, J=77.5 Hz), 28.17 (s), 14.08 (s), 11.97 (s), 6.92 (d, J=4.5 Hz); HRMS (ESI⁺) calculated for C₂₈H₃₀N₆O₅ [M+H]+: 530.5850, found 531.1412.

17e (15.6 mg, 41%). ¹H NMR (400 MHz, DMSO-d6) δ 8.27 (dd, J=12.0, 5.2 Hz, 1H), 7.87-7.71 (m, 1H), 7.29 (s, 1H), 7.05 (d, J=8.2 Hz, 1H), 6.74 (dd, J=8.3, 1.8 Hz, 1H), 6.35-6.06 (m, 1H), 4.59-4.50 (m, 2H), 4.13 (dd, J=14.0, 7.0 Hz, 2H), 3.76 (d, J=26.0 Hz, 2H), 3.46 (d, J=5.4 Hz, 1H), 3.30-3.09 (m, 6H), 3.00-2.90 (m, 1H), 2.84 (dd, J=14.5, 5.1 Hz, 1H), 1.93 (dd, J=18.0, 8.6 Hz, 1H), 1.75 (dt, J=12.8, 6.3 Hz, 1H), 1.49 (tt, J=7.7, 4.8 Hz, 1H), 1.21 (t, J=7.1 Hz, 3H), 0.81 (t, J=2.6 Hz, 1H), 0.79-0.76 (m, 2H), 0.73-0.71 (m, 2H); HRMS (ESI⁺) calculated for C₂₉H₃₃N₆O₄ [M+H]+: 529.2558, found 529.2688.

18a (54.6 mg, 75%)¹H NMR (400 MHz, DMSO-d6) δ 8.36 (s, 1H), 7.79-7.57 (m, 3H), 7.49 (s, 1H), 6.43 (d, J=39.3 Hz, 1H), 4.16-4.10 (m, 2H), 4.00 (d, J=35.6 Hz, 2H), 3.83 (s, 1H), 3.16 (d, J=35.7 Hz, 2H), 3.07-2.82 (m, 4H), 1.93 (s, 1H), 1.66 (s, 2H), 1.49 (ddd, J=12.4, 7.7, 4.9 Hz, 2H), 1.24-1.20 (m, 3H), 0.59 (d, J=81.9 Hz, 5H); HRMS (ESI⁺) calculated for C₂₉H₃₁F₄N₆O₃ [M+H]+: 587.2388, found 587.2401.

18b (29 mg, 77%)¹H NMR (400 MHz, DMSO) δ 8.35 (s, 1H), 7.60 (d, J=8.0 Hz, 3H), 7.28 (s, 1H), 6.47 (s, 1H), 4.18-4.08 (m, 2H), 3.94 (s, 1H), 3.83 (d, J=5.4 Hz, 2H), 3.28-2.55 (m, 7H), 1.78 (dd, J=11.9, 6.1 Hz, 1H), 1.72-1.59 (m, 2H), 1.41 (ddd, J=20.2, 10.4, 3.9 Hz, 2H), 1.22 (s, 3H), 0.73-0.44 (m, 4H); MS(ESI⁺) calculated for C₂₈H₃₁Cl₂N₆O₃⁺ [M+H]+: 569.1829 Found 569.8487.

18c (25 mg, 40%). ¹H NMR (400 MHz, MeOD) δ 8.20 (d, J=5.2 Hz, 1H), 8.00 (s, 1H), 7.90 (dd, J=12.6, 5.6 Hz, 3H), 7.59-7.52 (m, 2H), 7.44 (d, J=8.3 Hz, 1H), 6.35 (s, 1H), 4.28-4.20 (m, 2H), 3.93-3.87 (m, 1H), 3.54 (dd, J=80.1, 58.5 Hz, 3H), 3.28 (s, 1H), 3.07 (ddd, J=31.5, 20.3, 11.9 Hz, 4H), 1.52-1.35 (m, 9H), 1.34-1.30 (m, 6H), 0.91 (d, J=7.0 Hz, 2H); ¹³C NMR (101 MHz, DMSO-D6) δ 174.34 (s), 170.84 (s), 161.73 (s), 160.59 (d, J=4.9 Hz), 149.70 (s), 144.25 (s), 132.56 (t, J=4.8 Hz), 129.60 (s), 128.67 (s), 128.21 (d, J=6.4 Hz), 127.55 (s), 126.62 (d, J=19.6 Hz), 125.99 (s), 102.11 (s), 60.42 (s), 45.18 (s), 41.78 (s), 29.77 (s), 28.27 (d, J=19.3 Hz), 22.65 (s), 14.12 (s), 10.44 (s), 6.72 (d, J=30.3 Hz), 6.55-6.46 (m); HRMS (ESI⁺) calculated for C₃₂H₃₄N₆O₃ [M+H]+: 550.6630, found 551.7372.

18d (8.1 mg, 26%). ¹H NMR (400 MHz, MeOD) δ 8.24 (d, J=24.3 Hz, 1H), 6.92-6.84 (m, 3H), 6.26 (s, 1H), 6.01 (s, 2H), 4.26-4.18 (m, 2H), 4.04 (d, J=58.6 Hz, 2H), 3.85 (s, 1H), 3.21 (d, J=44.6 Hz, 2H), 3.08-2.95 (m, 2H), 2.10-1.94 (m, 2H), 1.80 (s, 2H), 1.54 (d, J=57.0 Hz, 3H), 1.34-1.29 (m, 6H), 0.92 (t, J=6.8 Hz, 2H), 0.87-0.74 (m, 2H). ¹³C NMR (101 MHz, DMSO-D6) δ 174.36 (s), 170.95 (s), 162.73 (s), 161.79 (s), 160.46 (s), 156.90 (s), 147.57 (s), 147.13 (s), 143.64 (s), 133.02 (s), 122.65 (s), 108.98-108.73 (m), 108.40 (d, J=45.5 Hz), 101.39 (d, J=2.5 Hz), 60.39 (s), 46.23 (s), 45.09 (s), 30.68 (s), 28.43 (s), 28.16 (s), 22.93 (s), 14.11 (s), 10.42 (s), 6.83 (d, J=3.6 Hz). HRMS (ESI+) calculated for C₂₉H₃₂N₆O₅ [M+H]+: 544.6120, found 545.9041.

18e (10.7 mg, 38%)¹H NMR (400 MHz, DMSO-d6) δ 8.24 (s, 1H), 7.50 (d, J=28.0 Hz, 1H), 7.26 (s, 1H), 7.03 (s, 1H), 6.73 (d, J=8.1 Hz, 1H), 6.15 (d, J=48.8 Hz, 1H), 4.55 (t, J=6.9 Hz, 2H), 4.13 (dd, J=7.1, 3.2 Hz, 2H), 3.79 (s, 1H), 3.17 (dd, J=16.0, 8.3 Hz, 6H), 2.94 (dd, J=17.9, 11.8 Hz, 2H), 2.83 (dd, J=16.8, 6.0 Hz, 2H), 1.96 (d, J=12.3 Hz, 1H), 1.78 (s, 3H), 1.24-1.19 (m, 4H), 0.71 (s, 3H), 0.60 (s, 1H), 0.49 (s, 1H); HIRMS (ESI+) calculated for C₃₀H₃₅N₆O₄ [M+H]+: 543.2714, found 543.3116.

19a (40 mg, 66%) ¹H NMR (400 MHz, DMSO-d6) δ 8.36 (s, 1H), 7.78-7.57 (m, 3H), 7.50 (s, 1H), 6.44 (d, J=39.0 Hz, 1H), 4.16-4.10 (m, 2H), 4.01 (d, J=34.9 Hz, 2H), 3.83 (s, 1H), 3.15 (d, J=38.2 Hz, 2H), 3.05-2.82 (m, 4H), 1.91 (s, 1H), 1.77 (dd, J=13.0, 7.2 Hz, 1H), 1.65 (s, 3H), 1.22 (d, J=4.8 Hz, 3H), 0.59 (d, J=81.6 Hz, 5H); HIRMS (ESI+) calculated for C₂₉H₃₁F₄N₆O₃ [M+H]+: 587.2388, found 587.2401.

19b (50 mg, 74%)¹H NMR (400 MHz, DMSO) δ 8.37 (s, 1H), 7.72-7.50 (m, 3H), 7.30 (s, 1H), 6.43 (d, J=47.6 Hz, 1H), 4.20-4.10 (m, 2H), 3.97 (d, J=6.3 Hz, 1H), 3.90-3.47 (m, 2H), 3.32-2.59 (m, 7H), 1.95 (s, 1H), 1.64 (dd, J=15.2, 11.2 Hz, 2H), 1.51-1.34 (m, 2H), 1.24 (d, J=5.6 Hz, 3H), 0.79-0.44 (m, 4H); MS(ESI+) calculated for C₂₈H₃₁Cl₂N₆O₃⁺ [M+H]+: 569.1829, found 569.9567.

19c (12.1 mg, 57%). ¹H NMR (400 MHz, DMSO-D6) δ 8.24 (s, 1H), 7.91 (dd, J=26.0, 19.5 Hz, 4H), 7.58-7.32 (m, 4H), 6.23 (d, J=66.0 Hz, 1H), 4.18-4.11 (m, 2H), 3.84 (s, 2H), 3.54-3.34 (m, 2H), 3.30-2.80 (m, 6H), 2.03-1.84 (m, 1H), 1.82-1.35 (m, 3H), 1.23 (td, J=7.1, 4.0 Hz, 3H), 0.93-0.77 (m, 1H), 0.63 (d, J=57.5 Hz, 4H); ¹³C NMR (101 MHz, DMSO-D6) δ 174.33 (s), 161.94-161.66 (m), 156.84 (s), 144.24 (s), 132.96-132.34 (m), 129.28 (s), 128.91 (s), 128.20 (d, J=4.4 Hz), 127.94-126.48 (m), 126.48-126.12 (m), 121.04 (s), 101.23 (s), 60.43 (s), 54.90 (s), 47.72 (s), 45.18 (s), 44.46 (s), 28.27 (d, J=19.1 Hz), 22.62 (s), 14.12 (s), 10.43 (s), 6.83 (d, J=7.9 Hz); IRMS (ESI+) calculated for C₃₂H₃₄N₆O₃ [M+H]+: 550.6630, found 551.8812.

19d (7.6 mg, 29%). ¹H NMR (400 MHz, DMSO-D6) δ 8.32-8.18 (m, 1H), 7.53 (d, J=39.0 Hz, 1H), 6.86 (dd, J=34.3, 7.9 Hz, 3H), 6.16 (t, J=25.4 Hz, 1H), 6.05 (s, 2H), 4.32-3.68 (m, 5H), 3.33 (s, 2H), 3.18-2.77 (m, 5H), 1.92 (d, J=26.5 Hz, 1H), 1.76 (d, J=23.6 Hz, 2H), 1.34 (dd, J=60.3, 29.5 Hz, 2H), 1.22 (t, J=5.8 Hz, 3H), 0.82-0.37 (m, 4H); 13C NMR (101 MHz, DMSO-D6) δ 174.36 (s), 170.94 (s), 161.79 (s), 160.43 (s), 149.35 (s), 147.57 (s), 147.13 (s), 143.63 (s), 132.99 (s), 129.34 (s), 122.65 (s), 108.65 (s), 108.18 (s), 101.34 (d, J=5.9 Hz), 60.39 (s), 49.10 (s), 46.79 (s), 45.08 (s), 41.91 (s), 28.43 (s), 28.16 (s), 26.76 (s), 23.00 (s), 14.11 (s), 10.42 (s), 6.72 (d, J=25.3 Hz); HRMS (ESI+) calculated for C₂₉H₃₂N₆O₅ [M+H]+: 544.6120, found 545.9761.

19e (11 mg, 41.7%)¹H NMR (400 MHz, DMSO-d6) δ 8.24 (s, 1H), 7.51 (d, J=34.7 Hz, 1H), 7.26 (s, 1H), 7.03 (s, 1H), 6.74 (t, J=9.9 Hz, 1H), 6.15 (d, J=50.0 Hz, 1H), 4.60-4.49 (m, 2H), 4.15-4.09 (m, 2H), 3.79 (s, 1H), 3.28-2.99 (m, 7H), 3.00-2.77 (m, 3H), 1.94 (dd, J=13.1, 6.1 Hz, 1H), 1.75 (d, J=25.4 Hz, 3H), 1.27-1.18 (m, 4H), 0.71 (s, 3H), 0.60 (s, 1H), 0.49 (s, 1H); HRMS (ESI⁺) calculated for C₃₀H₃₅N₆O₄ [M+H]+: 543.2714, found 543.2036.

### <Example 2>1-(2-(((S/R)-1-(cyclopropanecarbonyl)piperidine/pyrrolidine-3-yl)amino)pyrimidin-4-yl)-2-aryl-1,4,5,6-tetrahydrocyclopenta[d]imidazol-5-carboxamides (20a-e, 21a-e, 22a-e, and 23a-e)

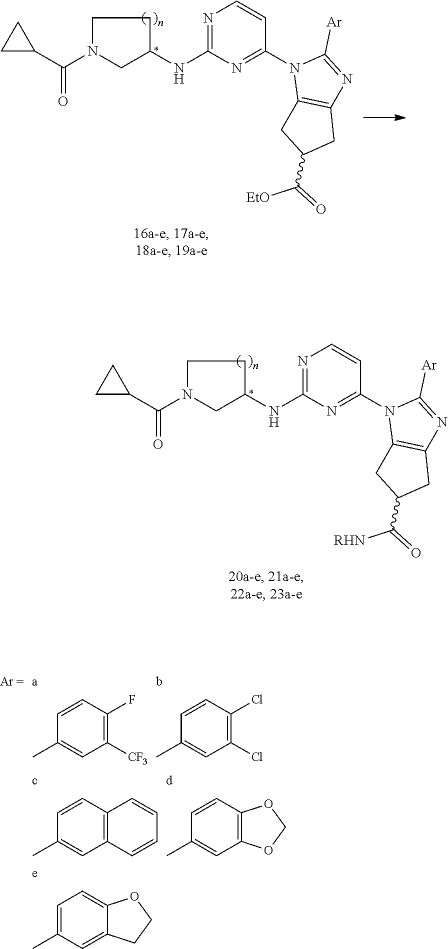

Compound 16a (23.3 mg, 0.040 mmol), which had been obtained in the previous experiment, was diluted in ammonia in 7N-methanol (0.91 mL) and then stirred at room temperature for 3 days. It was able to be confirmed that the initial ethyl ester develops into methyl ester, and an amide is formed from the methyl ester. After confirming the completion of the reactor, the resulting product was extracted with ethyl acetate (EtOAc), and the organic layer was washed with water and brine. After removing water with anhydrous magnesium sulfate (MgSO₄) and concentrating the solvent, the resulting solution was purified by column chromatography using MC and MeOH (MC:MeOH=20:1) as mobile phase, thereby obtaining Compound 20a (13.8 mg, 81.9%).

20a (13.8 mg, 81.9%). ¹H NMR (400 MHz, DMSO-d6) δ 8.38 (dd, J=9.4, 5.2 Hz, 1H), 7.92-7.66 (m, 3H), 7.59-7.40 (m, 2H), 6.94 (s, 1H), 6.51 (d, J=25.2 Hz, 1H), 3.72 (s, 1H), 3.69-3.50 (m, 2H), 3.40 (dd, J=17.8, 7.3 Hz, 1H), 3.29-3.21 (m, 1H), 3.08 (d, J=7.1 Hz, 2H), 2.95-2.84 (m, 1H), 2.77 (dd, J=14.6, 5.6 Hz, 1H), 1.83-1.65 (m, 2H), 1.61-1.43 (m, 1H), 0.85 (ddd, J=8.6, 5.2, 2.1 Hz, 1H), 0.76-0.64 (m, 4H); HRMS (ESI+) calculated for C₂₆H₂₆F₄N₇O₂ [M+H]+: 544.2079, found 544.2117.

20b (4 mg, 49%) ¹H NMR (400 MHz, DMSO) δ 8.38 (dd, J=13.5, 5.2 Hz, 1H), 7.83 (d, J=24.1 Hz, 1H), 7.66 (s, 1H), 7.63 (dd, J=8.4, 2.1 Hz, 1H), 7.45 (s, 1H), 7.31 (t, J=6.8 Hz, 1H), 6.94 (s, 1H), 6.50 (d, J=38.8 Hz, 1H), 3.77 (d, J=29.0 Hz, 1H), 3.66-3.59 (m, 1H), 3.55-3.44 (m, 2H), 3.40 (ddd, J=10.0, 6.9, 4.3 Hz, 2H), 3.07 (s, 2H), 2.93-2.84 (m, 1H), 2.76 (dd, J=14.1, 4.9 Hz, 1H), 1.83-1.67 (m, 2H), 1.50-1.40 (m, 1H), 0.76-0.65 (m, 4H); MS(ESI⁺) calculated for C₂₅H₂₆Cl₂N₇O₂⁺ [M+H]+: 526.1520, found 526.7123.

20c (8.4 mg, 68%). ¹H NMR (400 MHz, DMSO-D6) δ 8.34-8.24 (m, 1H), 7.99 (d, J=6.1 Hz, 1H), 7.90 (t, J=8.7 Hz, 3H), 7.72 (dd, J=25.4, 6.1 Hz, 1H), 7.59-7.35 (m, 4H), 6.94 (s, 1H), 6.40 (d, J=58.9 Hz, 1H), 3.65 (s, 2H), 3.58-3.35 (m, 2H), 3.16 (dd, J=28.0, 14.7 Hz, 4H), 2.97-2.75 (m, 2H), 1.65 (d, J=54.6 Hz, 2H), 1.23 (s, 1H), 0.68 (d, J=22.3 Hz, 4H); HRMS (ESI+) calculated for C₂₉H₂₉N₇O₂ [M+H]+: 507.5980, found 508.5647.

20d (26.7 mg, 92%). 1H NMR (400 MHz, DMSO-D6) δ 8.34-8.25 (m, 1H), 7.92 (d, J=4.6 Hz, 1H), 7.79 (d, J=24.3 Hz, 1H), 6.98-6.87 (m, 2H), 6.83 (dd, J=8.1, 1.4 Hz, 1H), 6.40-6.15 (m, 1H), 6.09-5.98 (m, 2H), 3.82-3.66 (m, 1H), 3.64-3.54 (m, 1H), 3.39 (d, J=40.4 Hz, 3H), 3.32-2.94 (m, 3H), 2.92-2.66 (m, 2H), 2.65-2.58 (m, 3H), 1.98 (d, J=3.5 Hz, 2H), 1.74-1.39 (m, 1H), 0.70 (dd, J=10.7, 7.4 Hz, 4H). HRMS (ESI+) calculated for C₂₆H₂₇N₇O₄ [M+H]+: 501.5470, found 502.5154.

20e (4.1 mg, 54%). ¹H NMR (400 MHz, DMSO-d6) δ 8.30-8.23 (m, 1H), 7.75 (d, J=24.3 Hz, 1H), 7.44 (s, 1H), 7.28 (s, 1H), 7.04 (d, J=8.3 Hz, 1H), 6.90 (s, 1H), 6.73 (dd, J=8.3, 2.4 Hz, 1H), 6.25 (d, J=41.7 Hz, 1H), 4.54 (dd, J=15.4, 8.2 Hz, 2H), 3.99 (s, 1H), 3.77 (d, J=8.5 Hz, 1H), 3.59 (s, 2H), 3.49 (t, J=4.2 Hz, 1H), 3.15 (t, J=7.3 Hz, 2H), 3.10-2.98 (m, 2H), 2.91-2.81 (m, 1H), 2.76-2.66 (m, 1H), 1.98-1.87 (m, 1H), 1.76 (dd, J=12.2, 5.9 Hz, 1H), 1.57 (d, J=24.5 Hz, 1H), 0.69 (t, J=19.8 Hz, 5H); HRMS (ESI+) calculated for C₂₇H₃₀N₇O₃ [M+H]+: 500.2405, found 500.2469.

21a (15 mg, 78.8%). ¹H NMR (400 MHz, DMSO-d6) δ 8.38 (dd, J=9.4, 5.2 Hz, 1H), 7.93-7.67 (m, 3H), 7.51 (dt, J=14.0, 5.5 Hz, 1H), 7.45 (s, 1H), 6.94 (s, 1H), 6.52 (d, J=25.5 Hz, 1H), 3.72 (s, 1H), 3.68-3.52 (m, 2H), 3.40 (dd, J=18.8, 9.9 Hz, 1H), 3.28 (s, 1H), 3.08 (d, J=7.1 Hz, 2H), 2.95-2.85 (m, 1H), 2.77 (dd, J=14.5, 5.5 Hz, 1H), 1.82-1.63 (m, 2H), 1.60-1.44 (m, 1H), 0.88-0.83 (m, 1H), 0.72-0.66 (m, 4H); HRMS (ESI+) calculated for C₂₆H₂₆F₄N₇O₂ [M+H]+: 544.2079, found 544.2117.

21b (17 mg, 80%)¹H NMR (400 MHz, DMSO) δ 8.37 (dd, J=13.4, 5.2 Hz, 1H), 7.83 (ddd, J=15.2, 8.2, 3.6 Hz, 1H), 7.68-7.60 (m, 2H), 7.45 (s, 1H), 7.31 (t, J=7.0 Hz, 1H), 6.94 (s, 1H), 6.50 (d, J=38.1 Hz, 1H), 3.74 (s, 1H), 3.62 (dd, J=14.7, 7.0 Hz, 2H), 3.51-3.41 (m, 1H), 3.27-2.70 (m, 6H), 1.74 (dd, J=17.6, 11.6 Hz, 2H), 1.55 (s, 1H), 0.76-0.63 (m, 4H); MS(ESI⁺) calculated for C₂₅H₂₆Cl₂N₇O₂ [M+H]+: 526.1520, found 526.8563.

21c (8 mg, 66%). ¹H NMR (400 MHz, DMSO-D6) δ 8.35-8.23 (m, 1H), 7.99 (d, J=6.2 Hz, 1H), 7.90 (t, J=8.8 Hz, 3H), 7.72 (dd, J=25.3, 6.3 Hz, 1H), 7.58-7.40 (m, 4H), 6.94 (s, 1H), 6.41 (d, J=54.2 Hz, 1H), 3.84-3.59 (m, 2H), 3.48 (d, J=45.9 Hz, 1H), 3.03 (dd, J=72.8, 9.5 Hz, 4H), 2.93-2.75 (m, 2H), 1.99 (s, 1H), 1.58 (s, 2H), 1.39-1.19 (m, 1H), 0.76-0.55 (m, 4H); ¹³C NMR (101 MHz, DMSO-D6) δ 175.87 (s), 170.33 (s), 162.17-161.47 (m), 156.91 (s), 144.80 (s), 132.84-132.03 (m), 129.74 (s), 128.66-128.41 (m), 127.57 (dd, J=47.3, 36.9 Hz), 126.60 (d, J=13.0 Hz), 102.13 (s), 59.74 (s), 51.13 (s), 46.12 (s), 43.59 (s), 30.83 (s), 28.91 (s), 28.68 (s), 14.08 (s), 6.93 (d, J=2.8 Hz); HRMS (ESI⁺) calculated for C₂₉H₂₉N₇O₂ [M+H]+: 507.5980, found 508.5647.

21d (18.3 mg, 65%). ¹H NMR (400 MHz, DMSO-D6) δ 8.36-8.25 (m, 1H), 7.79 (d, J=24.6 Hz, 1H), 7.44 (s, 1H), 6.98-6.87 (m, 3H), 6.82 (dd, J=8.1, 1.3 Hz, 1H), 6.31 (d, J=28.0 Hz, 1H), 6.09-5.98 (m, 2H), 3.75 (dd, J=20.4, 12.9 Hz, 1H), 3.60 (s, 1H), 3.52-3.37 (m, 2H), 3.17 (dt, J=112.0, 29.0 Hz, 4H), 2.80 (ddd, J=20.1, 14.4, 7.5 Hz, 2H), 2.10-1.57 (m, 3H), 0.75-0.63 (m, 4H); ¹³C NMR (101 MHz, DMSO-D6) δ 176.44 (s), 171.45 (s), 162.58-162.33 (m), 156.51 (s), 148.02 (s), 147.63 (s), 144.61 (s), 134.87 (s), 128.43 (s), 123.09 (s), 109.13 (s), 108.67 (s), 101.87 (d, J=4.5 Hz), 51.81 (s), 51.23 (s), 46.50 (s), 44.91 (s), 29.70-28.64 (m), 12.13 (s), 7.43 (d, J=5.9 Hz).

HRMS (ESI+) calculated for C₂₆H₂₇N₇O₄ [M+H]+: 501.5470, found 502.5154.

21e (7.1 mg, 53.6%). ¹H NMR (400 MHz, DMSO-d6) δ 8.30-8.23 (m, 1H), 7.75 (d, J=22.6 Hz, 1H), 7.42 (d, J=18.5 Hz, 1H), 7.28 (s, 1H), 7.04 (d, J=8.3 Hz, 1H), 6.91 (s, 1H), 6.73 (dd, J=8.3, 2.3 Hz, 1H), 6.25 (d, J=41.6 Hz, 1H), 4.54 (dd, J=16.0, 7.7 Hz, 2H), 4.02 (dd, J=14.2, 7.1 Hz, 1H), 3.74 (dd, J=22.2, 14.9 Hz, 1H), 3.56 (d, J=30.0 Hz, 2H), 3.50-3.46 (m, 1H), 3.16 (t, J=8.4 Hz, 2H), 3.04 (dd, J=16.9, 11.2 Hz, 2H), 2.85 (dd, J=14.2, 9.4 Hz, 1H), 2.73 (dd, J=14.5, 6.0 Hz, 1H), 1.95-1.84 (m, 1H), 1.75 (dt, J=12.4, 6.2 Hz, 1H), 1.60 (s, 1H), 0.73 (dd, J=12.5, 5.9 Hz, 5H); HRMS (ESI+) calculated for C₂₇H₃₀N₇O₃ [M+H]+: 500.2405, found 500.2830.

22a (10.5 mg, 47%). ¹H NMR (400 MHz, DMSO-d6) δ 8.36 (s, 1H), 7.77-7.39 (m, 5H), 6.94 (s, 1H), 6.45 (d, J=46.5 Hz, 1H), 4.02 (d, J=67.0 Hz, 2H), 3.68-3.58 (m, 1H), 3.02 (s, 2H), 2.90 (dd, J=24.6, 13.9 Hz, 2H), 2.74 (t, J=25.7 Hz, 2H), 1.89 (d, J=36.8 Hz, 1H), 1.62 (s, 3H), 1.46-1.33 (m, 1H), 0.77-0.41 (m, 5H).

HRMS (ESI+) calculated for C₂₇H₂₈F₄N₇O₂ [M+H]+: 558.2235, found 558.2905.

22b (20 mg, 55%)¹H NMR (400 MHz, MeOD) δ 8.38-8.27 (m, 1H), 7.62 (dd, J=3.3, 2.0 Hz, 1H), 7.56-7.53 (m, 1H), 7.29 (t, J=9.1 Hz, 1H), 6.59-6.40 (m, 1H), 4.12-3.99 (m, 1H), 3.83-3.75 (m, 1H), 3.30-3.13 (m, 3H), 3.10-2.88 (m, 3H), 1.84 (s, 2H), 1.57-1.40 (m, 2H), 1.31 (d, J=7.9 Hz, 2H), 0.92 (t, J=6.9 Hz, 2H), 0.85 (s, 1H), 0.79-0.56 (m, 2H). HRMS (ESI+) calculated for C₂₆H₂₇Cl₂N₇O₂ [M+H]+: 540.4490, found 540.4203.

22c (9 mg, 78%)¹H NMR (400 MHz, MeOD) δ 8.25 (d, J=35.1 Hz, 1H), 7.99 (d, J=6.0 Hz, 1H), 7.93-7.82 (m, 3H), 7.59-7.51 (m, 2H), 7.42 (d, J=8.2 Hz, 1H), 6.49 (d, J=55.7 Hz, 1H), 3.79 (ddd, J=18.4, 12.7, 6.3 Hz, 2H), 3.30-3.14 (m, 2H), 3.11-2.93 (m, 3H), 2.88 (s, 1H), 1.44 (d, J=29.3 Hz, 2H), 1.33 (d, J=14.7 Hz, 4H), 0.92 (t, J=6.9 Hz, 2H), 0.83 (s, 1H), 0.66 (d, J=47.0 Hz, 2H). HRMS (ESI+) calculated for C₃₀H₃₁N₇O₂ [M+H]+: 521.6250, found 522.5953.

22d (5.6 mg, 67%). ¹H NMR (400 MHz, MeOD) δ 8.24 (d, J=21.4 Hz, 1H), 6.95-6.81 (m, 3H), 6.25 (s, 1H), 6.01 (s, 2H), 3.95 (s, 1H), 3.81-3.72 (m, 1H), 3.29-2.82 (m, 6H), 1.80 (s, 2H), 1.62 (s, 2H), 1.31 (s, 2H), 0.92 (t, J=6.8 Hz, 2H), 0.87-0.81 (m, 1H), 0.67 (d, J=57.8 Hz, 2H). HRMS (ESI+) calculated for C₂₇H₂₉N₇O₄ [M+H]+: 515.5740, found 516.4862.

22e (7.3 mg, 55%)¹H NMR (400 MHz, DMSO-d6) δ 8.23 (s, 1H), 7.43 (s, 2H), 7.25 (s, 1H), 7.02 (s, 1H), 6.90 (s, 1H), 6.73 (t, J=8.7 Hz, 1H), 6.18 (dd, J=38.9, 33.6 Hz, 1H), 4.55 (dd, J=17.2, 8.2 Hz, 2H), 4.00 (dt, J=45.6, 22.8 Hz, 2H), 3.63-3.53 (m, 1H), 3.13 (dd, J=26.9, 19.0 Hz, 5H), 2.92-2.66 (m, 3H), 1.77 (s, 3H), 1.48 (s, 2H), 0.75-0.44 (m, 5H); HRMS (ESI+) calculated for C₂₈H₃₂N₇O₃ [M+H]+: 514.2561, found 514.2889.

23a (13.8 mg, 81.9%). ¹H NMR (400 MHz, DMSO-d6) δ 8.37 (s, 1H), 7.78-7.40 (m, 5H), 6.94 (s, 1H), 6.47 (d, J=47.9 Hz, 1H), 4.16-3.89 (m, 2H), 3.61 (dd, J=15.7, 7.9 Hz, 1H), 3.12-2.66 (m, 6H), 1.99-1.88 (m, 1H), 1.62 (s, 2H), 1.47-1.33 (m, 1H), 0.79-0.33 (m, 5H); HRMS (ESI+) calculated for C₂₇H₂₈F₄N₇O₂ [M+H]+: 558.2235, found 558.2545.

23b (20 mg, 74%)¹H NMR (400 MHz, DMSO) δ 8.36 (s, 1H), 7.48 (dd, J=93.0, 39.3 Hz, 5H), 6.94 (s, 1H), 6.58-6.28 (m, 1H), 4.22-3.85 (m, 2H), 3.69-3.56 (m, 1H), 3.23-2.64 (m, 7H), 1.92 (d, J=12.5 Hz, 1H), 1.63 (s, 2H), 1.35 (dd, J=5.3, 3.8 Hz, 2H), 0.78-0.45 (m, 4H); MS(ESI⁺) calculated for C₂₆H₂₈Cl₂N₇O₂ [M+H]+: 540.1676, found 540.4310.

23c (27 mg, 80%)¹H NMR (400 MHz, MeOD) δ 8.25 (d, J=35.1 Hz, 1H), 7.99 (d, J=6.0 Hz, 1H), 7.93-7.82 (m, 3H), 7.59-7.51 (m, 2H), 7.42 (d, J=8.2 Hz, 1H), 6.49 (d, J=55.7 Hz, 1H), 3.79 (ddd, J=18.4, 12.7, 6.3 Hz, 2H), 3.30-3.14 (m, 2H), 3.11-2.93 (m, 3H), 2.88 (s, 1H), 1.44 (d, J=29.3 Hz, 2H), 1.33 (d, J=14.7 Hz, 4H), 0.92 (t, J=6.9 Hz, 2H), 0.83 (s, 1H), 0.66 (d, J=47.0 Hz, 2H). HRMS (ESI+) calculated for C₃₀H₃₁N₇O₂ [M+H]+: 521.6250, found 522.5953.

23d (6 mg, 74%). 1H NMR (400 MHz, DMSO-D6) δ 8.26 (s, 1H), 7.48 (d, J=39.9 Hz, 2H), 6.89 (d, J=8.2 Hz, 3H), 6.82-6.78 (m, 1H), 6.34-6.10 (m, 1H), 6.04 (s, 2H), 4.45-3.68 (m, 2H), 3.63-3.54 (m, 1H), 3.02 (s, 4H), 2.78 (d, J=45.4 Hz, 3H), 1.97 (s, 1H), 1.76 (s, 2H), 1.40 (d, J=54.2 Hz, 2H), 0.74-0.43 (m, 4H); HRMS (ESI+) calculated for C₂₇H₂₉N₇O₄ [M+H]+: 515.5740, found 516.4862.

23e (6 mg, 35.5%). ¹H NMR (400 MHz, DMSO-d6) δ 8.23 (s, 1H), 7.43 (s, 2H), 7.26 (s, 1H), 7.02 (s, 1H), 6.90 (s, 1H), 6.73 (t, J=8.9 Hz, 1H), 6.18 (dd, J=39.9, 34.5 Hz, 1H), 4.56 (dd, J=16.9, 8.3 Hz, 2H), 4.15-3.83 (m, 2H), 3.63-3.53 (m, 1H), 3.13 (dd, J=26.9, 19.1 Hz, 5H), 2.92-2.63 (m, 3H), 1.76 (s, 3H), 1.47 (s, 2H), 0.78-0.39 (m, 5H). HRMS (ESI+) calculated for C₂₈H₃₂N₇O₃ [M+H]+: 514.2561, found 514.2899.

### <Example 3>1-(2-(((S/R)-1-(cyclopropanecarbonyl)piperidine/pyrrolidine-3-yl)amino)piperidin-4-yl)-N-methyl-2-aryl-1,4,5,6-tetrahydrocyclopenta[d]imidazol-5-carboxamides (24a-e, 25a-e, 26a-e, and 27a-e)

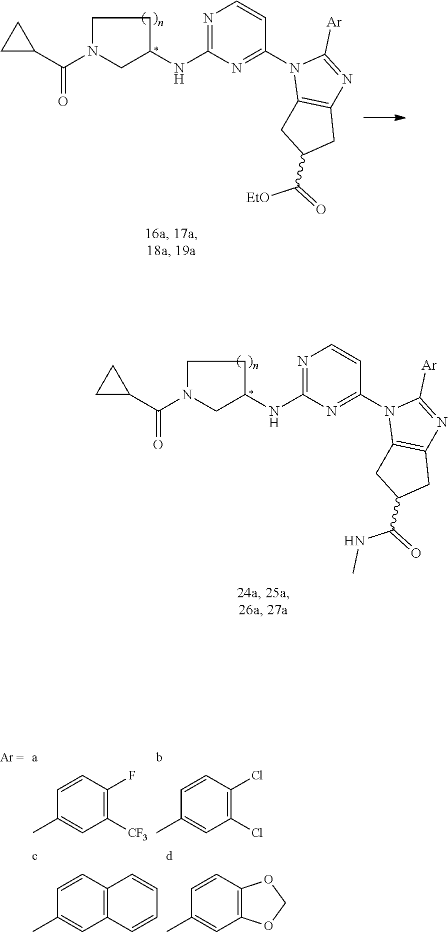

Synthesis was carried out using methylamine in 33% ethanol (1.75 mL) instead of an ammonia solution in the same synthesis method as above, and an organic layer was washed with water and brine. After removing water with anhydrous magnesium sulfate (MgSO₄) and concentrating the solvent, the resulting solution was purified by column chromatography using MC and MeOH (MC:MeOH=20:1) as mobile phase, thereby obtaining Compound 24.

24a (14.5 mg, 83.9%). ¹H NMR (400 MHz, DMSO-d6) δ 8.40-8.34 (m, 1H), 7.92 (dd, J=18.6, 6.7 Hz, 1H), 7.89-7.65 (m, 3H), 7.58-7.48 (m, 1H), 6.52 (d, J=28.1 Hz, 1H), 3.73 (d, J=6.3 Hz, 1H), 3.61 (dd, J=15.1, 7.2 Hz, 2H), 3.44-3.38 (m, 1H), 3.28-3.17 (m, 1H), 3.06 (d, J=8.1 Hz, 2H), 2.87 (dd, J=20.0, 11.0 Hz, 1H), 2.79-2.72 (m, 1H), 2.62 (d, J=4.4 Hz, 3H), 1.99-1.84 (m, 1H), 1.72 (s, 1H), 1.47 (dd, J=26.8, 19.8 Hz, 1H), 0.76-0.63 (m, 5H); HRMS (ESI+) calculated for C₂₇H₂₈F₄N₇O₂ [M+H]+: 558.2235, found 558.2545.

24b (17 mg, 91%)¹H NMR (400 MHz, DMSO) δ 8.37 (dd, J=13.7, 5.2 Hz, 1H), 7.94 (d, J=4.5 Hz, 1H), 7.87-7.74 (m, 1H), 7.68-7.60 (m, 2H), 7.31 (t, J=7.0 Hz, 1H), 6.60-6.36 (m, 1H), 3.74 (s, 1H), 3.67-3.40 (m, 3H), 3.27-2.69 (m, 6H), 2.62 (d, J=4.5 Hz, 3H), 2.02-1.87 (m, 1H), 1.82-1.65 (m, 2H), 0.73-0.67 (m, 4H); MS(ESI+) calculated for C₂₆H₂₈Cl₂N₇O₂⁺[M+H]+: 540.1676, found 540.5031.

24d (16 mg, 84%). 1H NMR (400 MHz, DMSO-D6) δ 8.33-8.26 (m, 4H), 7.91 (t, J=7.6 Hz, 4H), 7.79 (d, J=22.8 Hz, 4H), 6.96-6.87 (m, 8H), 6.82 (dd, J=8.1, 1.4 Hz, 4H), 6.31 (d, J=34.3 Hz, 3H), 6.07-5.99 (m, 8H), 3.76 (dt, J=26.3, 13.2 Hz, 4H), 3.65-3.54 (m, 6H), 3.33 (s, 12H), 3.30-2.90 (m, 14H), 2.90-2.65 (m, 9H), 2.65-2.58 (m, 12H), 2.07-1.52 (m, 13H), 0.75-0.64 (m, 17H). HRMS (ESI+) calculated for C₂₇H₂₉N₇O₄ [M+H]+: 515.5740, found 516.6663.

25a (19.4 mg, 99.4%)¹H NMR (400 MHz, DMSO-d6) δ 8.38 (dd, J=9.8, 5.2 Hz, 1H), 7.95 (d, J=4.5 Hz, 1H), 7.83-7.69 (m, 3H), 7.58-7.47 (m, 1H), 6.52 (d, J=28.5 Hz, 1H), 3.74 (s, 1H), 3.67-3.54 (m, 2H), 3.45-3.38 (m, 1H), 3.28-3.18 (m, 1H), 3.06 (d, J=7.7 Hz, 2H), 2.87 (dd, J=20.1, 10.9 Hz, 1H), 2.79-2.73 (m, 1H), 2.62 (d, J=4.5 Hz, 3H), 1.90 (dd, J=38.1, 15.8 Hz, 1H), 1.72 (s, 1H), 1.61-1.43 (m, 1H), 0.78-0.60 (m, 5H); HRMS (ESI+) calculated for C₂₇H₂₈F₄N₇O₂ [M+H]+: 558.2235, found 558.2545.

25b (5 mg, 67%)¹H NMR (400 MHz, DMSO) δ 8.39 (dd, J=13.8, 5.2 Hz, 1H), 7.97 (d, J=4.5 Hz, 1H), 7.69 (ddd, J=27.6, 18.8, 10.6 Hz, 3H), 7.33 (t, J=7.0 Hz, 1H), 6.52 (d, J=40.2 Hz, 1H), 3.77 (d, J=5.9 Hz, 1H), 3.55 (ddd, J=32.7, 17.2, 7.4 Hz, 3H), 3.28-2.72 (m, 6H), 2.64 (d, J=4.5 Hz, 3H), 1.97 (dd, J=21.1, 7.5 Hz, 1H), 1.82-1.67 (m, 2H), 0.72 (dd, J=9.8, 8.2 Hz, 4H); MS(ESI⁺) calculated for C₂₆H₂₈Cl₂N₇O₂⁺ [M+H]+: 540.1676, found 540.5031.

25d (27 mg, 92%). 1H NMR (400 MHz, DMSO-D6) δ 8.33-8.26 (m, 2H), 7.78 (d, J=24.4 Hz, 2H), 7.44 (s, 2H), 6.96-6.87 (m, 6H), 6.82 (dd, J=8.1, 1.4 Hz, 2H), 6.31 (d, J=29.3 Hz, 2H), 6.08-6.00 (m, 4H), 3.74 (dd, J=20.1, 12.7 Hz, 2H), 3.60 (s, 3H), 3.45 (dd, J=32.0, 12.1 Hz, 6H), 3.28-2.99 (m, 6H), 2.79 (ddd, J=20.1, 14.3, 7.4 Hz, 4H), 2.07-1.58 (m, 6H), 0.75-0.65 (m, 8H). 13C NMR (101 MHz, DMSO-D6) δ 176.46 (s), 171.50 (s), 162.43 (d, J=2.3 Hz), 144.79-144.50 (m), 126.51 (s), 123.10 (s), 109.12 (s), 108.68 (s), 101.87 (d, J=4.7 Hz), 51.77 (s), 51.23 (s), 46.49 (s), 44.92 (s), 29.77-29.10 (m), 12.48 (s), 7.44 (d, J=5.8 Hz); HRMS (ESI+) calculated for C₂₇H₂₉N₇O₄ [M+H]+: 515.5740, found 516.5222.

26a (17.6 mg, 77%). ¹H NMR (400 MHz, DMSO-d6) δ 8.36 (s, 1H), 7.93 (t, J=13.5 Hz, 1H), 7.79-7.55 (m, 3H), 7.50 (s, 1H), 6.45 (d, J=48.9 Hz, 1H), 4.02 (d, J=61.1 Hz, 2H), 3.68-3.58 (m, 1H), 3.11-2.75 (m, 6H), 2.62 (t, J=4.3 Hz, 3H), 1.95 (d, J=16.3 Hz, 1H), 1.62 (d, J=5.8 Hz, 2H), 1.50-1.27 (m, 2H), 0.75-0.43 (m, 5H); HRMS (ESI+) calculated for C₂₈H₃₀F₄N₇O₂ [M+H]+: 572.2392, found 572.2972.

26b (16 mg, 57%)¹H NMR (400 MHz, DMSO) δ 8.35 (s, 1H), 7.94 (d, J=4.5 Hz, 1H), 7.55 (d, J=44.7 Hz, 3H), 7.27 (s, 1H), 6.41 (d, J=53.6 Hz, 1H), 3.99 (d, J=38.5 Hz, 2H), 3.66-3.55 (m, 1H), 3.25-2.66 (m, 7H), 2.62 (t, J=4.6 Hz, 3H), 1.95 (d, J=12.2 Hz, 1H), 1.63 (s, 2H), 1.37 (dd, J=23.3, 15.5 Hz, 2H), 0.76-0.47 (m, 4H); MS(ESI+) calculated for C₂₇H₃₀Cl₂N₇O₂⁺[M+H]+: 554.1833, found 554.6538.

26c (9 mg, 87%). 1H NMR (400 MHz, DMSO-D6) δ 8.23 (s, 1H), 8.02-7.82 (m, 5H), 7.57-7.35 (m, 4H), 6.44-6.11 (m, 1H), 4.45-4.06 (m, 1H), 3.83 (s, 2H), 3.61 (dd, J=15.7, 7.8 Hz, 1H), 3.04 (d, J=45.2 Hz, 3H), 2.95-2.68 (m, 3H), 2.63 (t, J=4.0 Hz, 3H), 1.78 (dd, J=71.8, 52.5 Hz, 2H), 1.52-1.30 (m, 2H), 1.23 (s, 1H), 0.74-0.38 (m, 4H); HRMS (ESI+) calculated for C₃₁H₃₃N₇O₂ [M+H]+: 535.6520, found 536.5063.

27a (11.6 mg, 78%). ¹H NMR (400 MHz, DMSO-d6) δ 8.36 (s, 1H), 7.94 (d, J=4.4 Hz, 1H), 7.65 (d, J=63.4 Hz, 3H), 7.49 (s, 1H), 6.44 (d, J=49.8 Hz, 1H), 4.02 (d, J=62.4 Hz, 2H), 3.60 (dd, J=15.4, 7.8 Hz, 1H), 2.85 (ddd, J=33.8, 32.4, 25.2 Hz, 6H), 2.62 (t, J=4.3 Hz, 3H), 1.99-1.89 (m, 1H), 1.61 (s, 2H), 1.43 (d, J=7.5 Hz, 2H), 0.77-0.41 (m, 5H); HRMS (ESI+) calculated for C₂₈H₃₀F₄N₇O₂ [M+H]+: 572.2392, found 572.2972.

27b (19 mg, 86%)¹H NMR (400 MHz, DMSO) δ 8.35 (s, 1H), 7.94 (d, J=4.7 Hz, 1H), 7.56 (t, J=23.0 Hz, 3H), 7.27 (s, 1H), 6.41 (d, J=54.7 Hz, 1H), 3.99 (d, J=40.1 Hz, 2H), 3.66-3.55 (m, 1H), 3.23-2.66 (m, 7H), 2.62 (t, J=4.6 Hz, 3H), 1.95 (d, J=12.6 Hz, 1H), 1.63 (s, 2H), 1.51-1.29 (m, 2H), 0.63 (dd, J=74.5, 27.0 Hz, 4H); MS(ESI⁺) calculated for C₂₇H₃₀Cl₂N₇O₂⁺[M+H]+: 554.1833, found 554.4377.

27c (5.3 mg, 49%). 1H NMR (400 MHz, DMSO-D6) δ 8.23 (s, 1H), 7.95 (d, J=4.7 Hz, 1H), 7.88 (d, J=14.0 Hz, 2H), 7.73 (s, 3H), 7.57-7.50 (m, 1H), 7.40 (s, 1H), 6.26 (d, J=95.0 Hz, 1H), 3.67-3.57 (m, 1H), 3.50-3.40 (m, 3H), 2.95 (dd, J=72.1, 23.6 Hz, 3H), 2.66 (ddd, J=16.2, 12.2, 6.4 Hz, 5H), 2.00-1.84 (m, 2H), 1.31 (d, J=62.9 Hz, 3H), 0.78 (dt, J=115.4, 35.5 Hz, 4H). HRMS (ESI+) calculated for C₃₁H₃₃N₇O₂ [M+H]+: 535.6520, found 536.7582.

The synthesis methods according to Preparation Examples and Examples can be shown in Scheme 1 below.

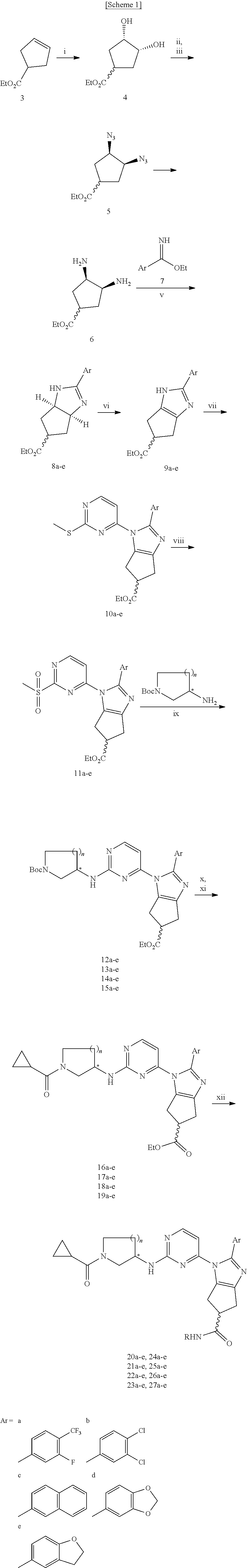

In Scheme 1, steps are as follows: (i) OsO₄, NMO, t-BuOH, THF, H₂O, room temperature, 2 hrs; (ii) MsCl, TEA, MC, room temperature, 2 hrs; (iii) NaN₃, DMF, 80° C., 12 hrs; (iv) Pd(OH)₂, H₂(g), MeOH, room temperature, 1 hr; (v) 7, aryl imidate, EtOH, 80° C., 5 hrs, (vi) oxalyl chloride, DMSO, TEA, MC, −78° C.->room temperature, 2 hrs; (vii) 10, Cs₂CO₃, DMF, 80° C., MW 1 hr; (viii) oxone, MeOH:H₂O=1:1, room temperature, 1 hr; (ix) 12, THF, 60° C., 5 hrs; (x) 4M-HCl in dioxane, room temperature, 1 hr; (xi) cyclopropanecarbonyl chloride, THF, 60° C., 5 hrs, xii) NH₃ in 7N MeOH.

### Experimental Example 1. Measurement of JNK3 Enzyme Activity

Changes in JNK3 enzyme activity due to treatment with the imidazole derivatives of Tables 1 to 5 below according to the present invention were confirmed through IC₅₀.

TABLE 1

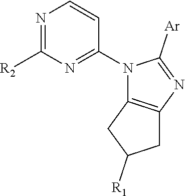

| Compound | | | | |
|---|---|---|---|---|
| No. | Ar | R₁ | R₂ | IC₅₀ (nM) |
| 16a | 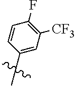 [SMILES: *c1ccc(F)c(C(F)(F)F)c1] | 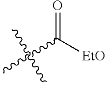 | 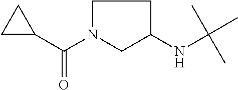 | + |
| 17a | | | 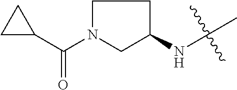 [SMILES: *N[C@@H]1CCN(C(=O)C2CC2)C1] | ++ |
| 18a | | | 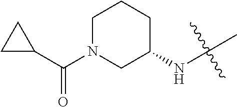 [SMILES: *N[C@H]1CCCN(C(=O)C2CC2)C1] | ++ |
| 19a | | | 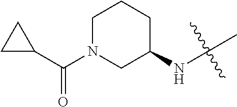 [SMILES: *N[C@@H]1CCCN(C(=O)C2CC2)C1] | ++ |
| 20a | | 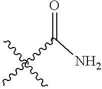 | 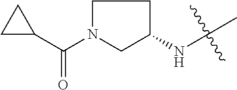 [SMILES: *N[C@H]1CCN(C(=O)C2CC2)C1] | + |
| 21a | | | 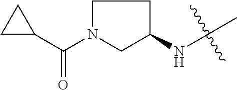 [SMILES: *N[C@@H]1CCN(C(=O)C2CC2)C1] | + |
| 22a | | | 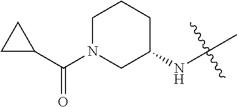 [SMILES: *N[C@H]1CCCN(C(=O)C2CC2)C1] | +++ |
| 23a | | | 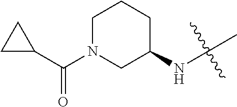 [SMILES: *N[C@@H]1CCCN(C(=O)C2CC2)C1] | + |
| 24a | | 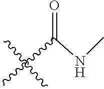 | 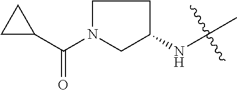 [SMILES: *N[C@H]1CCN(C(=O)C2CC2)C1] | + |
| 25a | | | 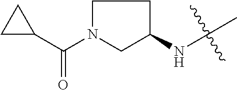 [SMILES: *N[C@@H]1CCN(C(=O)C2CC2)C1] | + |
| 26a | | | 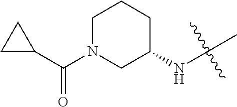 [SMILES: *N[C@H]1CCCN(C(=O)C2CC2)C1] | ++ |
| 27a | | | 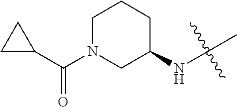 [SMILES: *N[C@@H]1CCCN(C(=O)C2CC2)C1] | + |

Activity = : >10 uM/+>1 uM/++: 0-0.03 uM/+++: <30 nM

TABLE 2

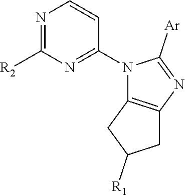

| Compound | | | | |
|---|---|---|---|---|
| No. | Ar | R₁ | R₂ | IC₅₀ (nM) |
| 16b | 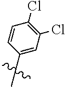 [SMILES: *c1ccc(Cl)c(Cl)c1] | 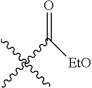 | 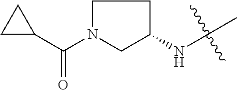 [SMILES: *N[C@H]1CCN(C(=O)C2CC2)C1] | +++ |
| 17b | | | 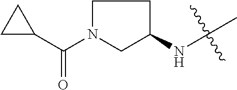 [SMILES: *N[C@@H]1CCN(C(=O)C2CC2)C1] | +++ |
| 18b | | | 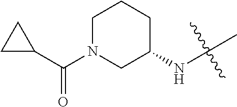 [SMILES: *N[C@H]1CCCN(C(=O)C2CC2)C1] | +++ |
| 19b | | | 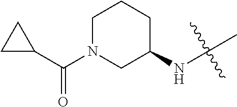 [SMILES: *N[C@@H]1CCCN(C(=O)C2CC2)C1] | +++ |
| 20b | | 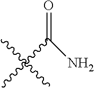 | 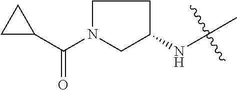 [SMILES: *N[C@H]1CCN(C(=O)C2CC2)C1] | +++ |
| 21b | | | 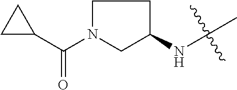 [SMILES: *N[C@@H]1CCN(C(=O)C2CC2)C1] | +++ |
| 22b | | | 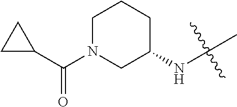 [SMILES: *N[C@H]1CCCN(C(=O)C2CC2)C1] | +++ |
| 23b | | | 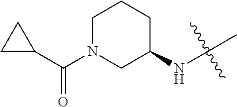 [SMILES: *N[C@@H]1CCCN(C(=O)C2CC2)C1] | +++ |
| 24b | | 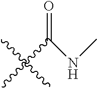 | 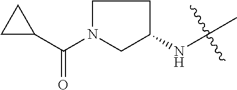 [SMILES: *N[C@H]1CCN(C(=O)C2CC2)C1] | +++ |
| 25b | | | 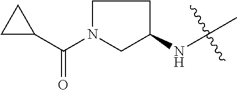 [SMILES: *N[C@@H]1CCN(C(=O)C2CC2)C1] | +++ |
| 26b | | | 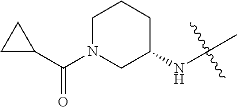 [SMILES: *N[C@H]1CCCN(C(=O)C2CC2)C1] | +++ |
| 27b | | | 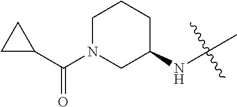 [SMILES: *N[C@@H]1CCCN(C(=O)C2CC2)C1] | +++ |

Activity = : >10 uM/+>1 uM/++: 0-0.03 uM/+++: <30 nM

TABLE 3

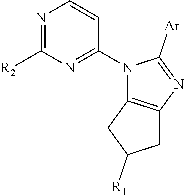

| Compound | | | | |
|---|---|---|---|---|
| No. | Ar | R₁ | R₂ | IC₅₀ (nM) |
| 16c | 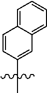 [SMILES: *c1ccc2ccccc2c1] | 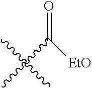 | 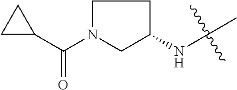 [SMILES: *N[C@H]1CCN(C(=O)C2CC2)C1] | +++ |
| 17c | | | 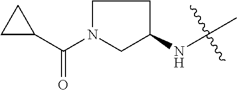 [SMILES: *N[C@@H]1CCN(C(=O)C2CC2)C1] | +++ |
| 18c | | | 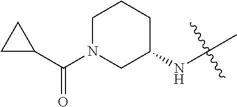 [SMILES: *N[C@H]1CCCN(C(=O)C2CC2)C1] | +++ |
| 19c | | | 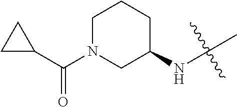 [SMILES: *N[C@@H]1CCCN(C(=O)C2CC2)C1] | +++ |
| 20c | | 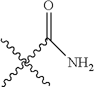 | 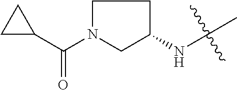 [SMILES: *N[C@H]1CCN(C(=O)C2CC2)C1] | +++ |
| 21c | | | 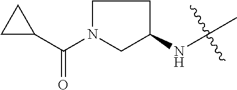 [SMILES: *N[C@@H]1CCN(C(=O)C2CC2)C1] | +++ |
| 22c | | | 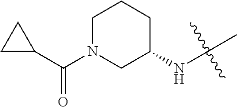 [SMILES: *N[C@H]1CCCN(C(=O)C2CC2)C1] | +++ |
| 23c | | | 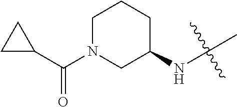 [SMILES: *N[C@@H]1CCCN(C(=O)C2CC2)C1] | +++ |
| 26c | | 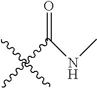 | 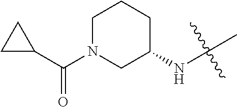 [SMILES: *N[C@H]1CCCN(C(=O)C2CC2)C1] | +++ |
| 27c | | | 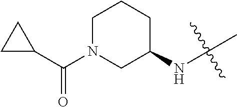 [SMILES: *N[C@@H]1CCCN(C(=O)C2CC2)C1] | +++ |

Activity = : >10 uM/+>1 uM/++: 0-0.03 uM/+++: <30 nM

TABLE 4

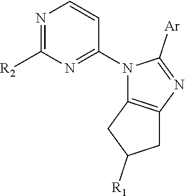

| Compound | | | | |
|---|---|---|---|---|
| No. | Ar | R₁ | R₂ | IC₅₀ (nM) |
| 16d | 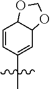 [SMILES: *C1=CC2OCOC2C=C1] | 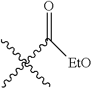 | 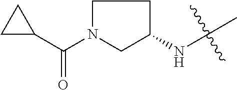 [SMILES: *N[C@H]1CCN(C(=O)C2CC2)C1] | + |
| 17d | | | 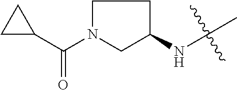 [SMILES: *N[C@@H]1CCN(C(=O)C2CC2)C1] | ++ |
| 18d | | | 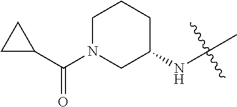 [SMILES: *N[C@H]1CCCN(C(=O)C2CC2)C1] | +++ |
| 19d | | | 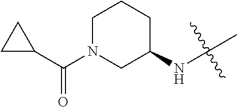 [SMILES: *N[C@@H]1CCCN(C(=O)C2CC2)C1] | +++ |
| 20d | | 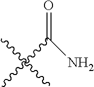 | 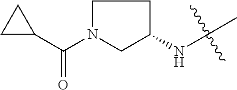 [SMILES: *N[C@H]1CCN(C(=O)C2CC2)C1] | + |
| 21d | | | 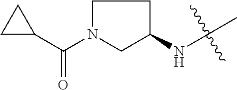 [SMILES: *N[C@@H]1CCN(C(=O)C2CC2)C1] | + |
| 22d | | | 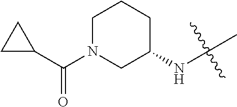 [SMILES: *N[C@H]1CCCN(C(=O)C2CC2)C1] | +++ |
| 23d | | | 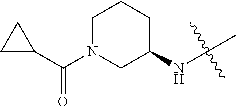 [SMILES: *N[C@@H]1CCCN(C(=O)C2CC2)C1] | ++ |
| 24d | | 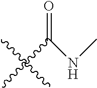 | 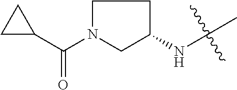 [SMILES: *N[C@H]1CCN(C(=O)C2CC2)C1] | + |
| 25d | | | 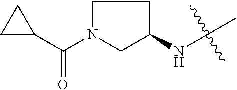 [SMILES: *N[C@@H]1CCN(C(=O)C2CC2)C1] | ++ |

Activity = : >10 uM/+>1 uM/++: 0-0.03 uM/+++: <30 nM

TABLE 5

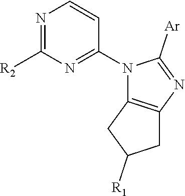

| Compound | | | | |
|---|---|---|---|---|
| No. | Ar | R₁ | R₂ | IC₅₀ (nM) |
| 16e | 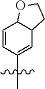 [SMILES: *C1=CC2CCOC2C=C1] | 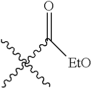 | 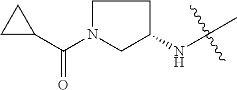 [SMILES: *N[C@H]1CCN(C(=O)C2CC2)C1] | + |
| 17e | | | 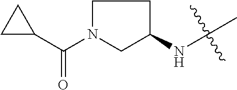 [SMILES: *N[C@@H]1CCN(C(=O)C2CC2)C1] | +++ |
| 18e | | | 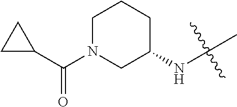 [SMILES: *N[C@H]1CCCN(C(=O)C2CC2)C1] | ++ |
| 19e | | | 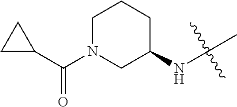 [SMILES: *N[C@@H]1CCCN(C(=O)C2CC2)C1] | +++ |
| 20e | | 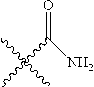 | 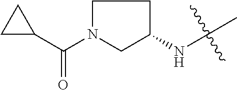 [SMILES: *N[C@H]1CCN(C(=O)C2CC2)C1] | ++ |
| 21e | | | 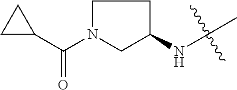 [SMILES: *N[C@@H]1CCN(C(=O)C2CC2)C1] | ++ |
| 22e | | | 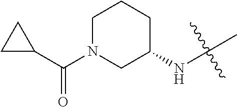 [SMILES: *N[C@H]1CCCN(C(=O)C2CC2)C1] | +++ |
| 23e | | | 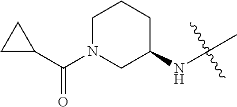 [SMILES: *N[C@@H]1CCCN(C(=O)C2CC2)C1] | +++ |
| 26e | | 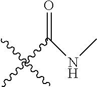 | 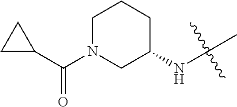 [SMILES: *N[C@H]1CCCN(C(=O)C2CC2)C1] | + |

Activity = : >10 uM/+>1 uM/++: 0-0.03 uM/+++: <30 nM

The IC₅₀ values of JNK3 of the synthesized compounds were measured and summarized in Tables 1 to 5.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as being illustrative and not restrictive in any aspect.

1. A compound of Formula 1 below or a pharmaceutically acceptable salt thereof: 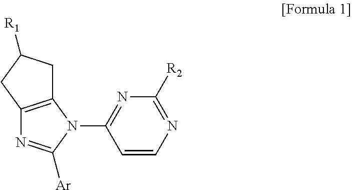
In Formula 1,
R₁ is selected from the group consisting of 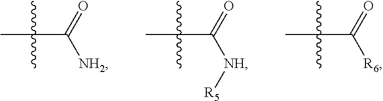
and C₁-C₆ alkyl,
R₂ is 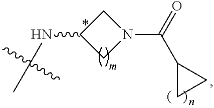
wherein n is an integer of 1 to 4,
m is an integer of 2 and 3, and
the carbon indicated by * is a chiral carbon,
Ar is selected from the group consisting of 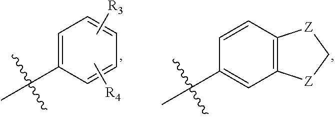
and naphthalenyl,
wherein R₃ and R₄ are each independently a halogen atom or halolalkyl,
R₅ is C₁-C₆ alkyl,
R₆ is alkoxy, and
Z is a carbon or oxygen atom.

2. The compound of claim 1, wherein the compound of Formula 1 has the structure of Formula 2 below: 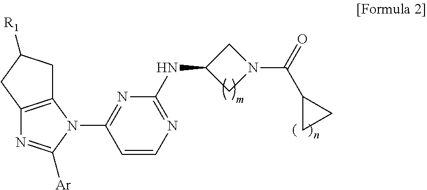
In Formula 2,
R₁ is selected from the group consisting of 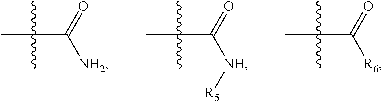
and C₁-C₆ alkyl,
n is an integer of 1 to 4,
m is an integer of 2 and 3,
Ar is selected from the group consisting of 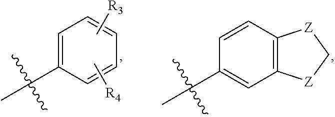
and naphthalenyl,
wherein R₃ and R₄ are each independently a halogen atom or haloalkyl,
R₅ is C₁-C₆ alkyl,
R₆ is alkoxy, and
Z is a carbon or oxygen atom.

3. The compound of claim 1, wherein the compound of Formula 1 has the structure of Formula 3 below: 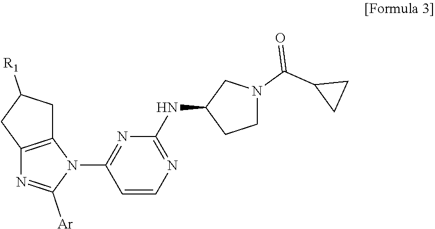
In Formula 3,
R₁ is selected from the group consisting of 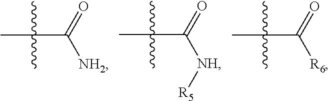
and C₁-C₆ alkyl,
Ar is selected from the group consisting of 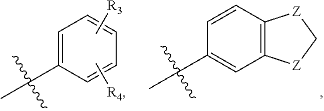
and naphthalenyl,
wherein R₃ and R₄ are each independently a halogen atom or haloalkyl,
R₅ is C₁-C₆ alkyl,
R₆ is alkoxy, and
Z is a carbon or oxygen atom.

4. The compound of claim 1, wherein the compound of Formula 1 has the structure of Formula 4 below: 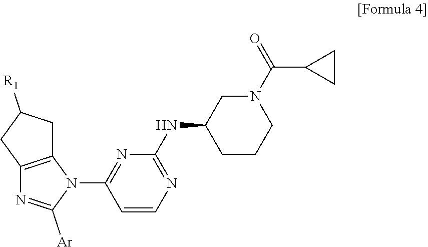
In Formula 4,
R₁ is selected from the group consisting of 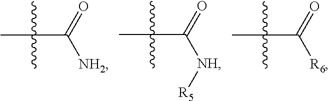
and C₁-C₆ alkyl,
Ar is selected from the group consisting of 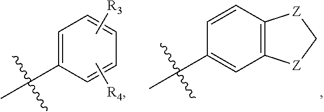
and naphthalenyl,
wherein R₃ and R₄ are each independently a halogen atom or haloalkyl,
R₅ is C₁-C₆ alkyl,
R₆ is alkoxy, and
Z is a carbon or oxygen atom.

5. The compound of claim 1, wherein the compound of Formula 1 has the structure of Formula 5 below: 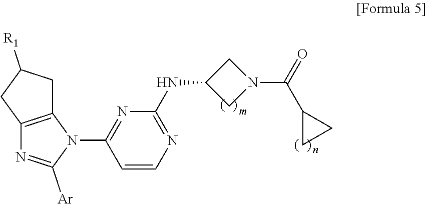
In Formula 5,
R₁ is selected from the group consisting of 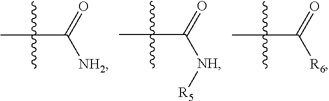
and C₁-C₆ alkyl,
n is an integer of 1 to 4,
m is an integer of 2 and 3,
Ar is selected from the group consisting of 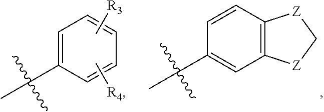
and naphthalenyl,
wherein R₃ and R₄ are each independently a halogen atom or haloalkyl,
R₅ is C₁-C₆ alkyl,
R₆ is alkoxy, and
Z is a carbon or oxygen atom.

6. The compound of claim 1, wherein the compound of Formula 1 has the structure of Formula 6 below: 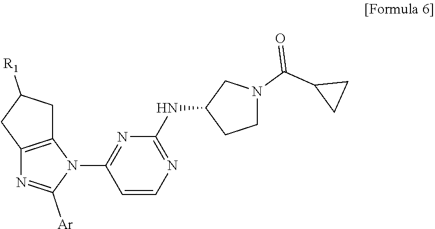
In Formula 6,
R₁ is selected from the group consisting of 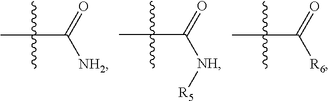
and C₁-C₆ alkyl,
Ar is selected from the group consisting of 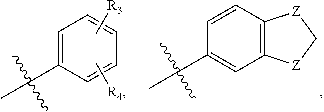
and naphthalenyl,
wherein R₃ and R₄ are each independently a halogen atom or haloalkyl,
R₅ is C₁-C₆ alkyl,
R₆ is alkoxy, and
Z is a carbon or oxygen atom.

7. The compound of claim 1, wherein the compound of Formula 1 has the structure of Formula 7 below: 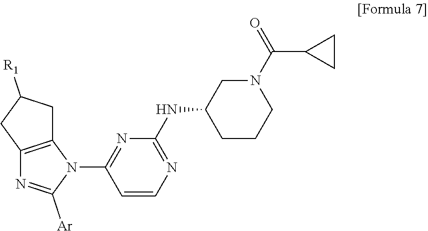
In Formula 7,
R₁ is selected from the group consisting of 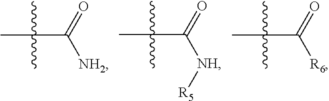
and C₁-C₆ alkyl,
Ar is selected from the group consisting of 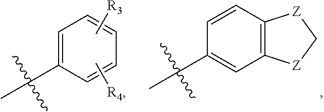
and naphthalenyl,
wherein R₃ and R₄ are each independently a halogen atom or haloalkyl,
R₅ is C₁-C₆ alkyl,
R₆ is alkoxy, and
Z is a carbon or oxygen atom.

8. The compound of claim 1, wherein the compound of Formula 1 is ethyl 1-(2-(((S)-1-(cyclopropanecarbonyl)pyrrolidine-3-yl)amino)pyrimidin-4-yl)-2-(4-fluoro-3-(trifluoromethyl)phenyl)-1,4,5,6-tetracyclopenta[d]imidazol-5-carboxylate;
ethyl 1-(2-(((R)-1-(cyclopropanecarbonyl)pyrrolidine-3-yl)amino)pyrimidin-4-yl)-2-(4-fluoro-3-(trifluoromethyl)phenyl)-1,4,5,6-tetracyclopenta[d]imidazol-5-carboxylate;
ethyl 1-(2-(((S)-1-(cyclopropanecarbonyl)piperidin-3-yl)amino)pyrimidin-4-yl)-2-(4-fluoro-3-(trifluoromethyl)phenyl)-1,4,5,6-tetracyclopenta[d]imidazol-5-carboxylate;
ethyl 1-(2-(((R)-1-(cyclopropanecarbonyl)piperidin-3-yl)amino)pyrimidin-4-yl)-2-(4-fluoro-3-(trifluoromethyl)phenyl)-1,4,5,6-tetracyclopenta[d]imidazol-5-carboxylate;
1-(2-(((S)-1-(cyclopropanecarbonyl)pyrrolidine-3-yl)amino)pyrimidin-4-yl)-2-(4-fluoro-3-(trifluoromethyl)phenyl)-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(4-fluoro-3-(trifluoromethyl)phenyl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(4-fluoro-3-(trifluoromethyl)phenyl)-1,4,5,6-tetracyclopenta[d]imidazol-5-carboxamide;
1-(2-(((R)-1-(cyclopropanecarbonyl)piperidin-3-yl)amino)pyrimidin-4-yl)-2-(4-fluoro-3-(trifluoromethyl)phenyl)-1,4,5,6-tetracyclopenta[d]imidazol-5-carboxamide;
1-(2-(((S)-1-(cyclopropanecarbonyl)pyrrolidine-3-yl)amino)pyrimidin-4-yl)-2-(4-fluoro-3-(trifluoromethyl)phenyl)-N-methyl-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(4-fluoro-3-(trifluoromethyl)phenyl)-N-methyl-1,4,5,6-tetracyclopenta[d]imidazol-5-carboxamide;
1-(2-(((S)-1-(cyclopropanecarbonyl)piperidin-3-yl)amino)pyrimidin-4-yl)-2-(4-fluoro-3-(trifluoromethyl)phenyl)-N-methyl-1,4,5,6-tetracyclopenta[d]imidazol-5-carboxamide;
1-(2-(((R)-1-(cyclopropanecarbonyl)piperidin-3-yl)amino)pyrimidin-4-yl)-2-(4-fluoro-3-(trifluoromethyl)phenyl)-N-methyl-1,4,5,6-tetracyclopenta[d]imidazol-5-carboxamide;
ethyl 1-(2-(((S)-1-(cyclopropanecarbonyl)pyrrolidine-3-yl)amino)pyrimidin-4-yl)-2-(3,4-dichlorophenyl)-1,4,5,6-tetracyclopenta[d]imidazol-5-carboxylate;
ethyl 1-(2-(((R)-1-(cyclopropanecarbonyl)pyrrolidine-3-yl)amino)pyrimidin-4-yl)-2-(3,4-dichlorophenyl)-1,4,5,6-tetracyclopenta[d]imidazol-5-carboxylate;
ethyl 1-(2-(((S)-1-(cyclopropanecarbonyl)piperidin-3-yl)amino)pyrimidin-4-yl)-2-(3,4-dichlorophenyl)-1,4,5,6-tetracyclopenta[d]imidazol-5-carboxylate;
ethyl 1-(2-(((R)-1-(cyclopropanecarbonyl)piperidin-3-yl)amino)pyrimidin-4-yl)-2-(3,4-dichlorophenyl)-1,4,5,6-tetracyclopenta[d]imidazol-5-carboxylate;
1-(2-(((S)-1-(cyclopropanecarbonyl)pyrrolidine-3-yl)amino)pyrimidin-4-yl)-2-(3,4-dichlorophenyl)-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(3,4-dichlorophenyl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(3,4-dichlorophenyl)-1,4,5,6-tetracyclopenta[d]imidazol-5-carboxamide;
1-(2-(((R)-1-(cyclopropanecarbonyl)piperidin-3-yl)amino)pyrimidin-4-yl)-2-(3,4-dichlorophenyl)-1,4,5,6-tetracyclopenta[d]imidazol-5-carboxamide;
1-(2-(((S)-1-(cyclopropanecarbonyl)pyrrolidine-3-yl)amino)pyrimidin-4-yl)-2-(3,4-dichlorophenyl)-N-methyl-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(3,4-dichlorophenyl)-N-methyl-1,4,5,6-tetracyclopenta[d]imidazol-5-carboxamide;
1-(2-(((S)-1-(cyclopropanecarbonyl)piperidin-3-yl)amino)pyrimidin-4-yl)-2-(3,4-dichlorophenyl)-N-methyl-1,4,5,6-tetracyclopenta[d]imidazol-5-carboxamide;
1-(2-(((R)-1-(cyclopropanecarbonyl)piperidin-3-yl)amino)pyrimidin-4-yl)-2-(4-fluoro-3-(trifluoromethyl)phenyl)-N-methyl-1,4,5,6-tetracyclopenta[d]imidazol-5-carboxamide;
ethyl 1-(2-(((S)-1-(cyclopropanecarbonyl)pyrrolidine-3-yl)amino)pyrimidin-4-yl)-2-(naphthalen-2-yl)-1,4,5,6-tetracyclopenta[d]imidazol-5-carboxylate;
ethyl 1-(2-(((R)-1-(cyclopropanecarbonyl)pyrrolidine-3-yl)amino)pyrimidin-4-yl)-2-(naphthalen-2-yl)-1,4,5,6-tetracyclopenta[d]imidazol-5-carboxylate;
ethyl 1-(2-(((S)-1-(cyclopropanecarbonyl)piperidin-3-yl)amino)pyrimidin-4-yl)-2-(naphthalen-2-yl)-1,4,5,6-tetracyclopenta[d]imidazol-5-carboxylate;
ethyl 1-(2-(((R)-1-(cyclopropanecarbonyl)piperidin-3-yl)amino)pyrimidin-4-yl)-2-(naphthalen-2-yl)-1,4,5,6-tetracyclopenta[d]imidazol-5-carboxylate;
1-(2-(((S)-1-(cyclopropanecarbonyl)pyrrolidine-3-yl)amino)pyrimidin-4-yl)-2-(naphthalen-2-yl)-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(naphthalen-2-yl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(naphthalen-2-yl)-1,4,5,6-tetracyclopenta[d]imidazol-5-carboxamide;
1-(2-(((R)-1-(cyclopropanecarbonyl)piperidin-3-yl)amino)pyrimidin-4-yl)-2-(naphthalen-2-yl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(naphthalen-2-yl)-N-methyl-1,4,5,6-tetracyclopenta[d]imidazol-5-carboxamide;
1-(2-(((R)-1-(cyclopropanecarbonyl)piperidin-3-yl)amino)pyrimidin-4-yl)-2-(4-fluoro-3-(naphthalen-2-yl)-N-methyl-1,4,5,6-tetracyclopenta[d]imidazol-5-carboxamide;
ethyl 2-(benzo[d][1,3]dioxol-5-yl)-1-(2-(((S)-1-(cyclopropanecarbonyl)pyrrolidine-3-yl)amino)pyrimidin-4-yl)-1,4,5,6-tetracyclopenta[d]imidazol-5-carboxylate;
ethyl 2-(benzo[d][1,3]dioxol-5-yl)-1-(2-(((R)-1-(cyclopropanecarbonyl)pyrrolidine-3-yl)amino)pyrimidin-4-yl)-1,4,5,6-tetracyclopenta[d]imidazol-5-carboxylate;
ethyl 2-(benzo[d][1,3]dioxol-5-yl)-1-(2-(((S)-1-(cyclopropanecarbonyl)piperidin-3-yl)amino)pyrimidin-4-yl)-1,4,5,6-tetracyclopenta[d]imidazol-5-carboxylate;
ethyl 2-(benzo[d][1,3]dioxol-5-yl)-1-(2-(((R)-1-(cyclopropanecarbonyl)piperidin-3-yl)amino)pyrimidin-4-yl)-1,4,5,6-tetracyclopenta[d]imidazol-5-carboxylate;
2-(benzo[d][1,3]dioxol-5-yl)-1-(2-(((S)-1-(cyclopropanecarbonyl)pyrrolidine-3-yl)amino)pyrimidin-4-yl)-1,4,5,6-tetracyclopenta[d]imidazol-5-carboxamide;
2-(benzo[d][1,3]dioxol-5-yl)-1-(2-(((R)-1-(cyclopropanecarbonyl)pyrrolidine-3-yl)amino)pyrimidin-4-yl)-1,4,5,6-tetracyclopenta[d]imidazol-5-carboxamide;
2-(benzo[d][1,3]dioxol-5-yl)-1-(2-(((S)-1-(cyclopropanecarbonyl)piperidin-3-yl)amino)pyrimidin-4-yl)-1,4,5,6-tetracyclopenta[d]imidazol-5-carboxamide;
2-(benzo[d][1,3]dioxol-5-yl)-1-(2-(((S)-1-(cyclopropanecarbonyl)piperidin-3-yl)amino)pyrimidin-4-yl)-1,4,5,6-tetracyclopenta[d]imidazol-5-carboxamide;
2-(benzo[d][1,3]dioxol-5-yl)-1-(2-(((S)-1-(cyclopropanecarbonyl)pyrrolidine-3-yl)amino)pyrimidin-4-yl)-N-methyl-1,4,5,6-tetracyclopenta[d]imidazol-5-carboxamide;
2-(benzo[d][1,3]dioxol-5-yl)-1-(2-(((S)-1-(cyclopropanecarbonyl)pyrrolidine-3-yl)amino)pyrimidin-4-yl)-N-methyl-1,4,5,6-tetracyclopenta[d]imidazol-5-carboxamide;
ethyl 1-(2-(((S)-1-(cyclopropanecarbonyl)pyrrolidine-3-yl)amino)pyrimidin-4-yl)-2-(2,3-dihydrobenzofuran-5-yl)-1,4,5,6-tetracyclopenta[d]imidazol-5-carboxylate;
ethyl 1-(2-(((R)-1-(cyclopropanecarbonyl)pyrrolidine-3-yl)amino)pyrimidin-4-yl)-2-(2,3-dihydrobenzofuran-5-yl)-1,4,5,6-tetracyclopenta[d]imidazol-5-carboxylate;
ethyl 1-(2-(((S)-1-(cyclopropanecarbonyl)piperidin-3-yl)amino)pyrimidin-4-yl)-2-(2,3-dihydrobenzofuran-5-yl)-1,4,5,6-tetracyclopenta[d]imidazol-5-carboxylate;
ethyl 1-(2-(((R)-1-(cyclopropanecarbonyl)piperidin-3-yl)amino)pyrimidin-4-yl)-2-(2,3-dihydrobenzofuran-5-yl)-1,4,5,6-tetracyclopenta[d]imidazol-5-carboxylate;
1-(2-(((S)-1-(cyclopropanecarbonyl)pyrrolidine-3-yl)amino)pyrimidin-4-yl)-2-(2,3-dihydrobenzofuran-5-yl)-1,4,5,6-tetracyclopenta[d]imidazol-5-carboxamide;
1-(2-(((R)-1-(cyclopropanecarbonyl)pyrrolidine-3-yl)amino)pyrimidin-4-yl)-2-(2,3-dihydrobenzofuran-5-yl)-1,4,5,6-tetracyclopenta[d]imidazol-5-carboxamide;
1-(2-(((S)-1-(cyclopropanecarbonyl)piperidin-3-yl)amino)pyrimidin-4-yl)-2-(2,3-dihydrobenzofuran-5-yl)-1,4,5,6-tetracyclopenta[d]imidazol-5-carboxamide;
1-(2-(((R)-1-(cyclopropanecarbonyl)piperidin-3-yl)amino)pyrimidin-4-yl)-2-(2,3-dihydrobenzofuran-5-yl)-1,4,5,6-tetracyclopenta[d]imidazol-5-carboxamide; or
1-(2-(((S)-1-(cyclopropanecarbonyl)piperidin-3-yl)amino)pyrimidin-4-yl)-2-(2,3-dihydrobenzofuran-5-yl)-N-methyl-1,4,5,6-tetracyclopenta[d]imidazol-5-carboxamide.

9. A pharmaceutical composition for use in preventing or treating a degenerative brain disease, comprising:
the imidazole derivative of claim 1, or a pharmaceutically acceptable salt thereof as an active ingredient.

10. The pharmaceutical composition of claim 9, wherein the degenerative brain disease is Alzheimer's dementia, or Parkinson's disease.

11. The pharmaceutical composition of claim 9, wherein the composition inhibits the activity of c-Jun N-terminal kinase 3 (JNK3).

12. A method for use in preventing or treating a degenerative brain disease comprising a step of administering the compound of Formula 1 of claim 1, an isomer thereof, a hydrate thereof, a solvate thereof, or a pharmaceutically acceptable salt thereof to a subject in need thereof in a pharmaceutically effective amount.

13. The method for use in preventing or treating a degenerative brain disease of claim 12, wherein the disease is Alzheimer's dementia, or Parkinson's disease.

14. The method for use in preventing or treating a degenerative brain disease of claim 12, wherein the composition inhibits the activity of c-Jun N-terminal kinase 3 (JNK3).

15. A pharmaceutical composition for use in preventing or treating a degenerative brain disease, comprising:
the imidazole derivative of claim 2, or a pharmaceutically acceptable salt thereof as an active ingredient.

16. A pharmaceutical composition for use in preventing or treating a degenerative brain disease, comprising:
the imidazole derivative of claim 3, or a pharmaceutically acceptable salt thereof as an active ingredient.

17. A pharmaceutical composition for use in preventing or treating a degenerative brain disease, comprising:
the imidazole derivative of claim 4, or a pharmaceutically acceptable salt thereof as an active ingredient.

18. A pharmaceutical composition for use in preventing or treating a degenerative brain disease, comprising:
the imidazole derivative of claim 5, or a pharmaceutically acceptable salt thereof as an active ingredient.

19. A pharmaceutical composition for use in preventing or treating a degenerative brain disease, comprising:
the imidazole derivative of claim 6, or a pharmaceutically acceptable salt thereof as an active ingredient.

20. A pharmaceutical composition for use in preventing or treating a degenerative brain disease, comprising:
the imidazole derivative of claim 8, or a pharmaceutically acceptable salt thereof as an active ingredient.
